(19) United States
(12) Patent Application Publication
(10) Pub. No.: US 2012/0094986 A1
(43) Pub. Date: Apr. 19, 2012

(54) ANTICANCER DERIVIATIVES, PREPARATION THEREOF, AND THERAPEUTIC USE THEREOF

(75) Inventors: Jérôme Arigon, Saint-Gely-Du-Fesc (FR); Claude Bernhart, Saint-Gely-Du-Fesc (FR); Monsif Bouaboula, Juvignac (FR); Romain Combet, Montpellier (FR); Sandrine Hilairet, Paris (FR); Samir Jegham, Montferrier-Sur-Lez (FR)

(73) Assignee: SANOFI-AVENTIS, Paris (FR)

(21) Appl. No.: 13/258,847

(22) PCT Filed: Mar. 22, 2010
(86) PCT No.: PCT/FR2010/050512

§ 371 Date: Dec. 1, 2011

(30) Foreign Application Priority Data
Mar. 24, 2009 (FR) 09/01366

(51) Int. Cl. A61K 31/444; C07D 417/14; A61P 35/00; C07D 401/14; A61K 31/4545; C07D 401/12; A61K 31/541

(52) U.S. Cl. 514/227.8; 546/262; 514/332; 544/58.2; 546/194; 514/318

(57) ABSTRACT

The invention relates to nicotinamide derivatives which can be used as anticancer drugs.

The present invention relates to novel anticancer derivatives, to the compositions containing them and to the therapeutic use thereof, in particular as anticancer agents. The invention also relates to the process for preparing these compounds and also to some of the intermediate products.

### PRIOR ART

WO 99/31064 describes anticancer compounds of formula (A):

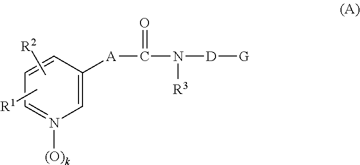

in which A represents in particular an alkylene group in which a methylene unit can be substituted with an O, S, C═O, NH, SO or SO₂ fragment in any position not adjacent to the amide unit —C(═O)—NR₃—. D represents an alkylene, alkenylene or alkinylene group containing at least 3 carbon atoms, in which from 1 to 3 methylene unit(s) may be substituted with an O, S, C═O, NH, SO or SO₂ fragment. G represents in particular the group —(CR₉R₁₀)ₘ—R₈ in which m is 0 or 1, R₉ and R₁₀ can represent a hydrogen atom or an alkyl group, and R₈ represents an aralkyl group or an aromatic or heteroaromatic group which is monocyclic, possibly containing from 1 to 3 heteroatoms (N, O or S) or a bicyclic or tricyclic aromatic group. R₈ may be optionally substituted with: halogen, —CN, alkyl, fluoroalkyl, cycloalkyl, aralkyl, aryl, —OH, hydroxyalkyl, alkoxy (—O-alkyl), aryloxy (—O-aryl), mercapto (—SH), alkylthio (—S-alkyl), arylthio (—S-aryl), carboxyl (—COOH), carboxyalkyl(-alkyl-COOH), carboxyalkenyl (-alkenyl-COOH), alkoxycarbonyl (—COOalkyl), nitro (—NO₂), amino (—NH₂), aminoalkyl(-alkyl-NH₂), monoalkylamino (—NHalkyl), dialkylamino (—N(alkyl)₂). This application describes neither the urea bond —NH—C(═O)—NH— nor the substitution of the nucleus comprising Z and Z′ with the groups —C(═O)NHR₂ and —NR₁R′₁.

US 2006/0040956 describes anticancer compounds of formula (B):

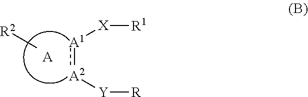

in which A can represent the ring

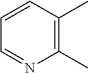

and R₂ represents a group chosen from H, halogen, —OR₃, oxo, —SR₃, —CO₂R₃, —COR₃, —CONR₃R₃, —NR₃R₃, —SO₂NR₃R₃, —NR₃COOR₃, —NR₃COR₃, cycloalkyl, optionally substituted phenyl, alkyl, cyano, nitro, etc.

WO 00/50399 describes biologically active compounds, that can be used as anticancer agents, of formula (C):

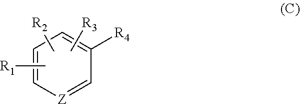

in which Z represents CH or N and R₁₋₄ represent carbohydrate groups. In particular, R₄ may be the group

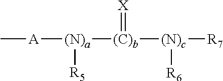

in which A represents a single bond or a carbohydrate group and R₇ represents a carbohydrate group. The 8 compounds described are characterized by one of the following units:

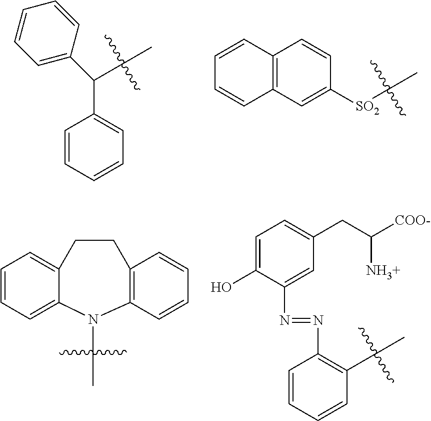

None of these documents describes or suggests the compounds of the invention.

### DESCRIPTION OF THE INVENTION

### Definitions Used

In the context of the present invention:
the term “halogen atom” is intended to mean: a fluorine, chlorine, bromine or iodine atom;
the term “alkyl group” is intended to mean: a saturated aliphatic hydrocarbon-based group containing from 1 to 10 carbon atoms (advantageously from 1 to 6 carbon atoms) of formula CₙH₂ₙ₊₁—, obtained by removing a hydrogen atom from an alkane. The alkyl group may be linear or branched. By way of examples, mention may be made of methyl, ethyl, propyl, isopropyl, butyl, isobutyl, tert-butyl, pentyl, 2,2-dimethylpropyl and hexyl groups;
the term “alkylene group” is intended to mean: a divalent group of molecular formula —CH₂ₙ—, obtained by removing two hydrogen atoms from an alkane on two different carbon atoms of said alkane;
the term “alkoxy group” is intended to mean: an —O-alkyl group, where the alkyl group is as defined above;
the term “cycloalkyl group” is intended to mean: a cyclic alkyl group containing between 3 and 8 carbon atoms, all the carbon atoms being involved in the cyclic structure. By way of examples, mention may be made of cyclopropyl or cyclopentyl groups;
the term “heterocycloalkyl group” is intended to mean: a cycloalkyl group comprising at least one heteroatom (O, S, N) involved in the ring and linked to the carbon atoms forming the ring. By way of examples, mention may be made of pyrrolidinyl, piperidinyl, piperazinyl or N—(C₁-C₄)alkylpiperazinyl, azepanyl, thiomorpholinyl, 1-oxo-thiomorpholinyl and 1,1-dioxo-thiomorpholinyl groups.

According to a 1st aspect, a subject of the present invention is a compound of formula (I):

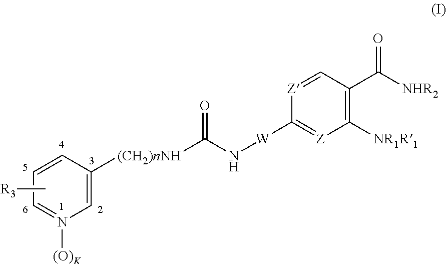

in which:
when Z and Z′ represent N or CH, W represents a —(C₁-C₄)alkylene-CH₂CH₂—, —(C₁-C₄)alkylene-CH═CH— or —(C₁-C₄)alkylene-C═C— group in which the (C₁-C₄)alkylene group is linked to the —C(═O)—NH— group;
when Z and Z′ represent respectively N and CH, W represents:
   a 1,4-cyclohexylene group

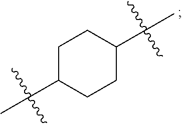

a —(CH₂)₁₋₄CRR′-group in which the (CH₂)₁₋₄ group is linked to the —C(═O)—NH-group and R and R′ represent a fluorine atom or a (C₁-C₄)alkyl group or form, together with the carbon atom to which they are attached, a cyclopropyl group;
k is an integer equal to 0 or 1;
n is an integer equal to 0, 1 or 2;
R₁ represents a hydrogen atom, a (C₁-C₆)alkyl group, a (C₃-C₆)cycloalkyl group or a phenyl group optionally substituted with a trifluoromethyl group;
R′₁ represents a hydrogen atom or a (C₁-C₆)alkyl group;
R₂ represents:
   a (C₃-C₆)cycloalkyl group;
   a (C₁-C₆)alkyl group optionally substituted with:
      one or more hydroxyl or (C₁-C₄)alkoxy groups;
      an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-, and
   being optionally substituted with one or more substituent(s), which may be identical to or different from one another when there are several thereof, chosen from an —OH; (C₁-C₄)alkoxy or (C₁-C₄)alkyl group;
R₃ represents at least one substituent of the pyridine nucleus, chosen from a hydrogen or fluorine atom, a (C₁-C₄)alkyl group, a cyano or —NR_{c}R_{d} in which R_{c} and R_{d} represent a hydrogen atom or a (C₁-C₄)alkyl group.

Z and Z′ represent N or CH. More particularly, Z and Z′ can represent respectively N and CH; CH and CH or N and N:

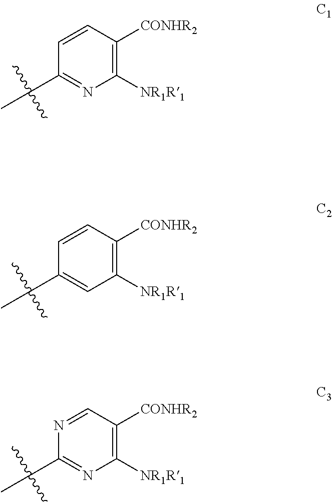

For the three rings C₁₋₃, W can represent a —(C₁-C₄)alkylene-CH₂CH₂—, —(C₁-C₄)alkylene-CH═CH— or —(C₁-C₄)alkylene-C≡C— group, the (C₁-C₄)alkylene group being linked to the —C(═O)—NH— group. The —(C₁-C₄)alkylene-CH═CH— group may be E or Z.

For the ring C₁, W can represent a 1,4-cyclohexylene group (cis or trans) or else a —(CH₂)₁₋₄CRR′-group in which the (CH₂)₁₋₄ group is linked to the —C(═O)—NH— group and R and R′ represent a fluorine atom or a (C₁-C₄)alkyl group or form, together with the carbon atom to which they are attached, a cyclopropyl group.

n is an integer that may be equal to 0, 1 or 2. Preferably, n is equal to 1.

R₁ represents a hydrogen atom, a (C₁-C₆)alkyl group, a (C₃-C₆)cycloalkyl group, for example cyclopropyl, or a phenyl group optionally substituted with a trifluoromethyl group.

R′₁ represents a hydrogen atom or the (C₁-C₆)alkyl group. More particularly, R′₁ represents a hydrogen atom. R₁ and/or R′₁ may be chosen from those described in Table I.

R₂ represents:
a (C₃-C₆)cycloalkyl group, for instance a cyclopropyl or cyclopentyl group;
a (C₁-C₆)alkyl group optionally substituted with:
   one or more —OH or (C₁-C₄)alkoxy, for example methoxy, group(s);
   an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-q more particularly represents 1 or 2.

The heterocycloalkyl group formed by Rₐ and R_{b} may, for example, be a

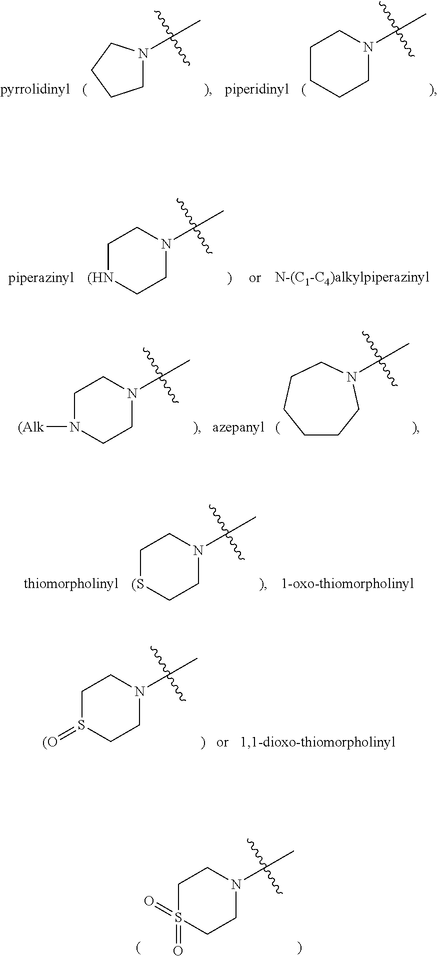

group.

The heterocycloalkyl group formed by Rₐ and R_{b} may be optionally substituted with one or more substituent(s), which may be identical to or different from one another when there are several thereof, chosen from: —OH; (C₁-C₄)alkoxy: for example, methoxy; (C₁-C₄)alkyl: for example, methyl. Thus, the substituted heterocycloalkyl may be a

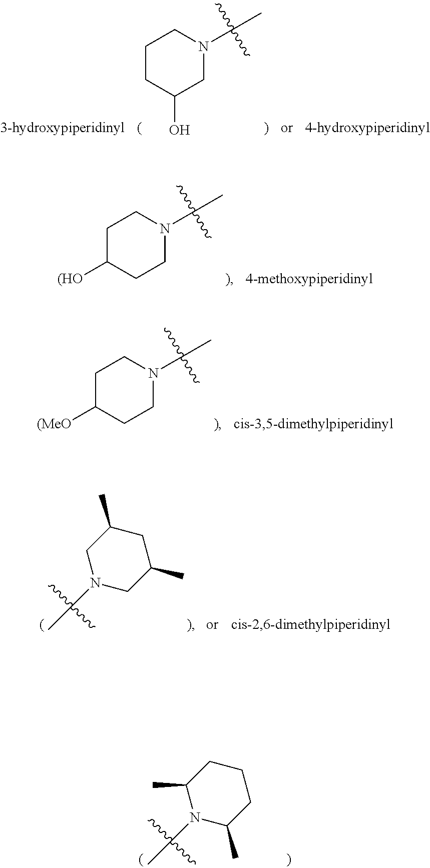

group.

R₂ may be chosen from one of those described in Table I.

The pyridine nucleus may comprise from 1 to 4 substituents R₃ chosen from a hydrogen or fluorine atom, a (C₁-C₄)alkyl group, a cyano or —NR_{c}R_{d} in which R_{c} and R_{d} represent a hydrogen atom or a (C₁-C₄)alkyl group. R₃ may be chosen from those described in Table I. Preferably, R₃ is in position 5 and/or 6 on the pyridine nucleus. Preferably, the number of substituents R₃ is equal to 1 and/or R₃ is in position 5 or 6 on the pyridine nucleus as is represented below:

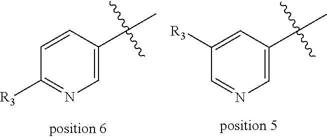

R₃ is even more preferably in position 6. Preferably, R₃ represents a hydrogen atom, —NH₂ or CN.

The subgroup of formula (I′) is differentiated:

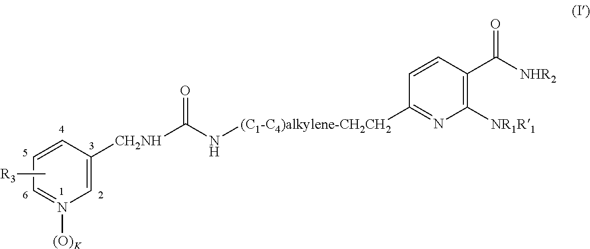

in which k, R₁, R′₁, R₂, R₃ are as defined above.

The subgroup of formula (I″) is differentiated:

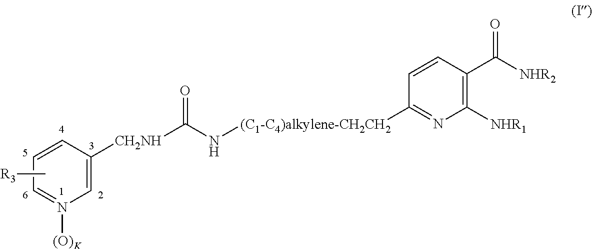

in which k is 0 or 1, R₁ represents a (C₁-C₄)alkyl group, R₂ represents a (C₁-C₆)alkyl group optionally substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, and R₃ is positioned in position 5 or 6.

The (C₁-C₄)alkylene group of W denotes more particularly the —(CH₂)₁₋₄-group.

The subgroup of formula (I′″) is differentiated:

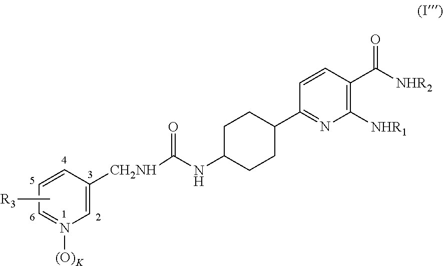

in which k is 0 or 1, R₁ represents a phenyl group optionally substituted with a trifluoromethyl group, R₂ represents a (C₁-C₆)alkyl group substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, and R₃ is positioned in position 5 or 6.

Among the compounds which are subjects of the invention, mention may be made of those of Table I.

The compounds of the invention, including the exemplified compounds, can exist in the form of bases or of addition salts with acids. Such addition salts are also part of the invention. These salts are advantageously prepared with pharmaceutically acceptable acids, but the salts of other acids that are useful, for example, for purifying or isolating the compounds are also part of the invention. The compounds according to the invention may also exist in the form of hydrates or of solvates, i.e. in the form of associations or combinations with one or more molecules of water or with a solvent. Such hydrates and solvates are also part of the invention.

The compounds may comprise one or more asymmetrical carbon atoms. They may therefore exist in the form of enantiomers or of diastereoisomers. These enantiomers and diastereoisomers, and also mixtures thereof, are part of the invention.

According to the present invention, the N-oxides of the compounds comprising an amine or a nitrogen atom are also part of the invention.

According to a 2nd aspect, a subject of the invention is the process for preparing the compounds of the invention and also some of the reaction intermediates.

Preparation of the Compounds of Formula I for which W═(C₁-C₄)alkylene-C≡C—

These compounds can be prepared according to Scheme 1, 2 or 3.

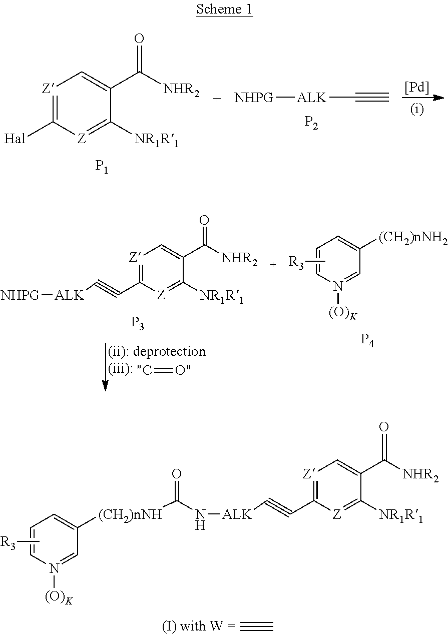

In stage (i), Sonogashira coupling is carried out between P₁ and P₂ so as to obtain P₃. Hal represents a halogen (chlorine, bromine, iodine) atom, ALK represents a (C₁-C₄)alkylene group and PG represents an amine-function-protecting group, for example BOC. The coupling is carried out in the presence of a palladium (in the oxidation state (0) or (II)) complex in a solvent in a basic medium. The palladium complex may, for example, be Pd(PPh₃)₄, PdCl₂(PPh₃)₂, Pd(OAc)₂, PdCl₂(dppf) or bis(di-tert-butyl(4-dimethylaminophenyl)phosphine)dichloropalladium(II).

A copper (I) salt, such as cuprous chloride, is generally required as a cocatalyst of the palladium complex. However, it has recently been discovered that certain catalytic systems do not require a copper salt, for instance the system Pd₂(dba)₃, P(t-Bu)₃ or Et₃N in THF (Eur. J. Org. Chem. 2000, 3679).

It is preferable to carry out the process in a deoxygenated medium so as to preserve the catalytic system when the latter is sensitive to oxygen.

The coupling is carried out in the presence of a basic medium, which may be, for example, K₂CO₃, NaHCO₃, Et₃N, K₃PO₄, Ba(OH)₂, NaOH, KF, CsF, Cs₂CO₃, etc. The coupling may be carried out in a mixture of a polar solvent, for example DMF. The temperature is between 50 and 120° C. The reaction time may in certain cases exceed be long (see conditions of Ex.1.3.).

Further details on the Sonogashira coupling (Scheme 1 of Chem. Rev.) and on the operating conditions and the palladium complexes, copper salts and bases that can be used, will be found in: Chem. Rev. 2007, 107(3), 874; Tetrahedron Lett. 2007, 48, 7129-7133; K. Sonogashira in “Metal-Catalyzed Cross-coupling Reactions”, 1998, eds.: F. Diederich, P. J. Stang, Wiley-VCH, Weinheim, ISBN 3-527-29421-X.

In stage (ii), P₃ is deprotected, for example by treatment in an acidic medium when PG represents BOC. In stage (iii), P₃ and P₄ are reacted in the presence of an agent for introducing the “C═O” unit (for example, phosgene, triphosgene or N,N′-disuccinimidyl carbonate DSC). The reaction for introducing “C═O” is preferably carried out in the presence of a base such as, for example, triethylamine and at a temperature of between −5° C. and ambient temperature. The solvent may be THF.

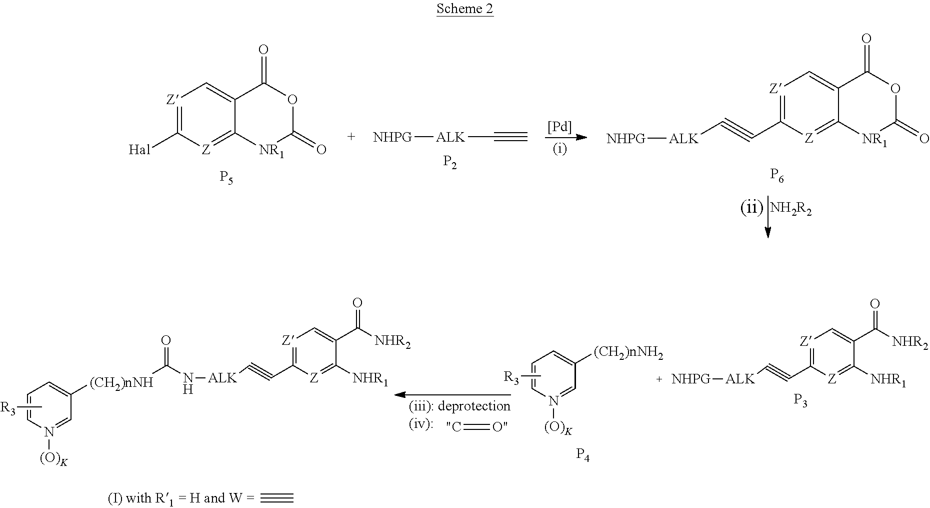

According to Scheme 2, in stage (i), Sonogashira coupling is carried out between P₅ and P₂ so as to obtain P₆, and then, in stage (ii), P₆ is reacted with the amine R₂NH₂ so as to obtain P₃. Stages (iii) and (iv) are similar to stages (ii) and (iii) of Scheme 1.

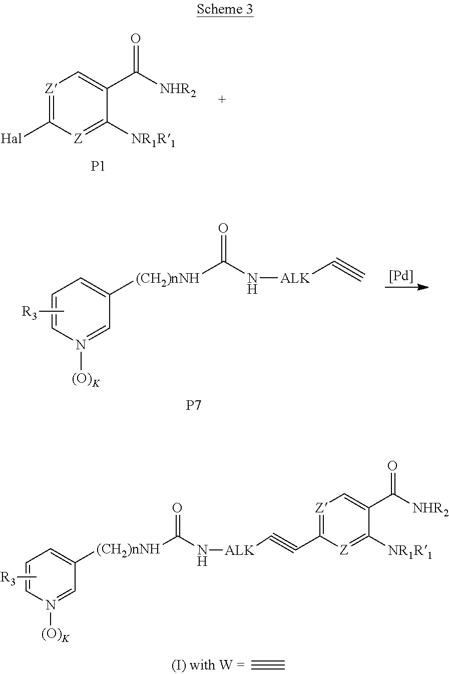

According to Scheme 3, Sonogashira coupling is carried out between P₁ and P₇ under the conditions described in detail above.

Preparation of the Compounds of Formula I for which W═(C₁-C₄)alkylene-CH₂CH₂—

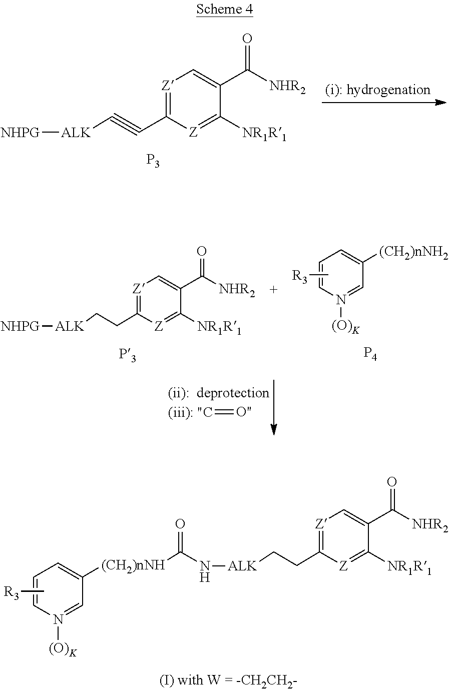

According to Scheme 4 (which is similar to Scheme 1), P′₃ and P₄ are coupled in the presence of the agent for introducing the “C═O” unit. P′₃ is obtained by hydrogenation of P₃.

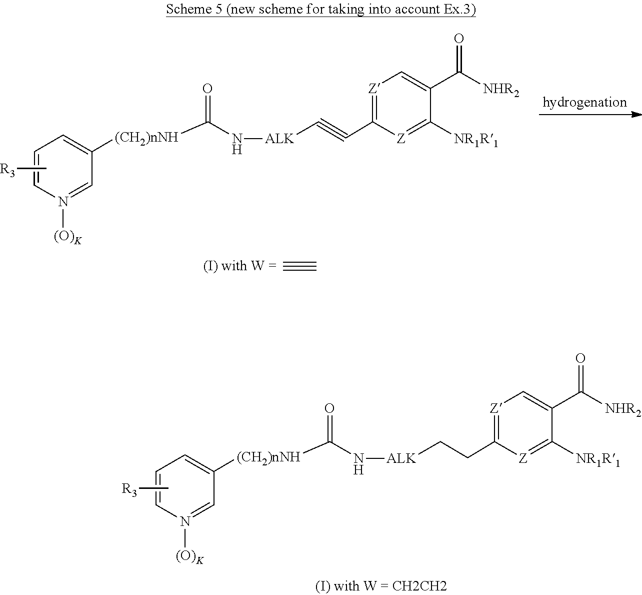

An alternative consists in hydrogenating a compound of formula (I) for which W═(C₁-C₄)alkylene-C≡C—.

The hydrogenation in Schemes 4 and 5 is carried out in the presence of hydrogen and of a metal catalyst, for example palladium deposited on a solid support (for example, Pd/C). The hydrogenation may be carried out, for example, at ambient temperature, with hydrogen under a pressure of the order of 1 atm in the presence of palladium-on-charcoal, for a period of the order of 20-30 min. See, for example, the conditions of Ex.3.6. Other techniques for hydrogenating —C≡C— bonds exist and are known to those skilled in the art.

Preparation of the Compounds of Formula I for which W═(C₁-C₄)alkylene-CH═CH—

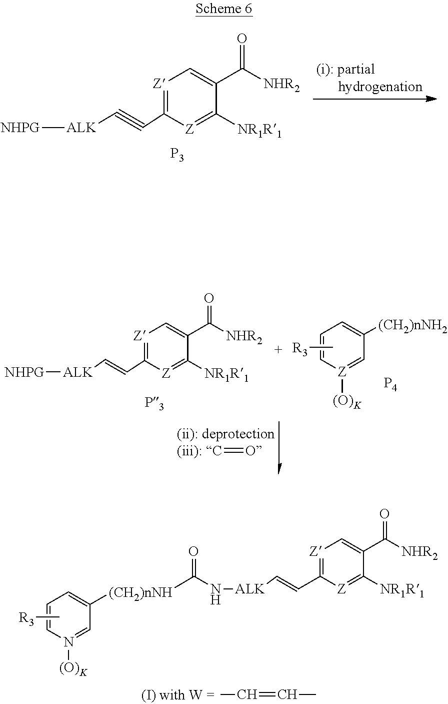

According to Scheme 6 (which is similar to Scheme 1), P″₃ and P₄ are coupled in the presence of the agent for introducing the “C═O” unit. P″₃ is obtained by partial hydrogenation of P₃.

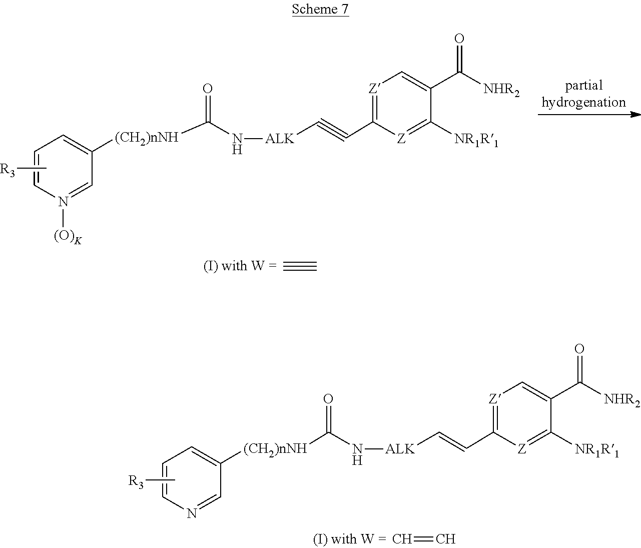

An alternative consists in partially hydrogenating a compound of formula (I) for which W═(C₁-C₄)alkylene-C≡C—.

For Schemes 6 and 7, the term “partial hydrogenation” is intended to mean the fact of not completely hydrogenating all the —C≡C— bonds of the starting product; to do this, use may, for example, be made of a deactivated or partially deactivated hydrogenation catalyst, for instance the Lindlar catalyst (it is typically Pd/CaCO₃ deactivated with quinoline or lead acetate). The partial hydrogenation thus results in a mixture of the starting product and of the products comprising the double and the triple bond which can then be separated by chromatography. Depending on the type of hydrogenation, it is possible to obtain a double bond, one of the forms of which (Z or E) is favoured.

Preparation of the Compounds of Formula (I) for which Z═N, Z═CH and W=1,4-cyclohexylene

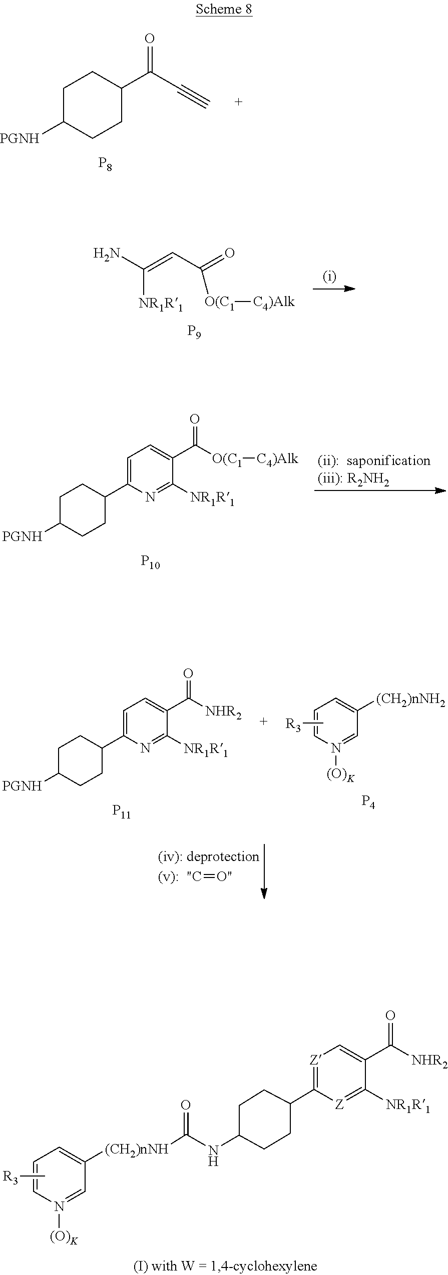

In stage (i), P₈ and the acrylate P₉ are reacted so as to obtain P₁₀ (Bohlmann-Rahtz reaction; in this respect, see Bagley, Synthesis 2007, 2459). In stage (ii), the ester function of P₁₀ is saponified and the resulting product is acidified so as to obtain the acid equivalent of P₁₀, which subsequently reacts, in stage (iii), with the amine R₂NH₂ so as to obtain P₁₁ (amidation). An acid activator such as BOP or (O-benzotriazol-1-yl)-N,N,N′,N′-tetramethyluronium tetrafluoroborate (TBTU) can be used. Stages (iv) and (v) are similar to stages (ii) and (iii) of Scheme 1.

Preparation of the Compounds of Formula (I) for which Z═N, Z═CH and W═(CH₂)₁₋₄CRR′—

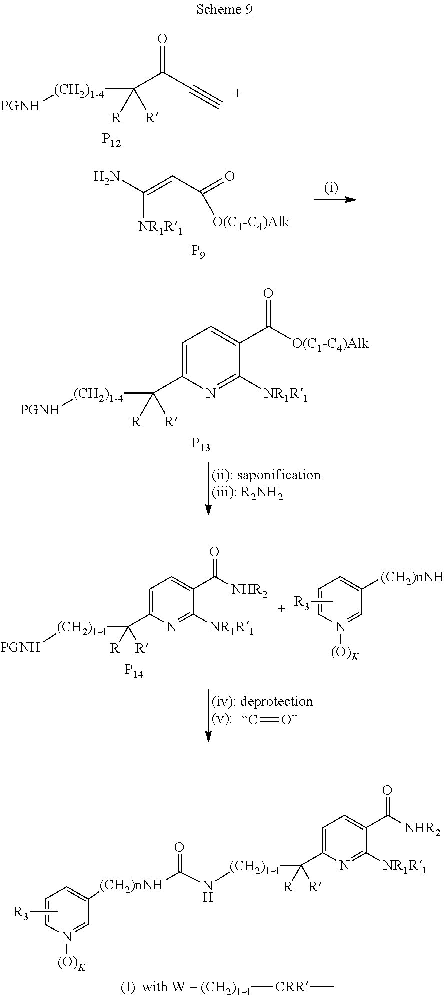

In stage (i), P₁₂ and the acrylate P₉ are reacted so as to obtain P₁₃ (Bohlmann-Rahtz reaction). In stage (ii), the ester function of P₁₃ is saponified and the resulting product is acidified so as to obtain the acid equivalent of P₁₃, which subsequently reacts, in stage (iii), with the amine R₂NH₂ so as to obtain P₁₄ (amidation). An acid activator such as BOP or (O-benzotriazol-1-yl)-N,N,N′,N′-tetramethyluronium tetrafluoroborate (TBTU) can be used. Stages (iv) and (v) are similar to stages (ii) and (iii) of Scheme 1.

### Preparation of P₁

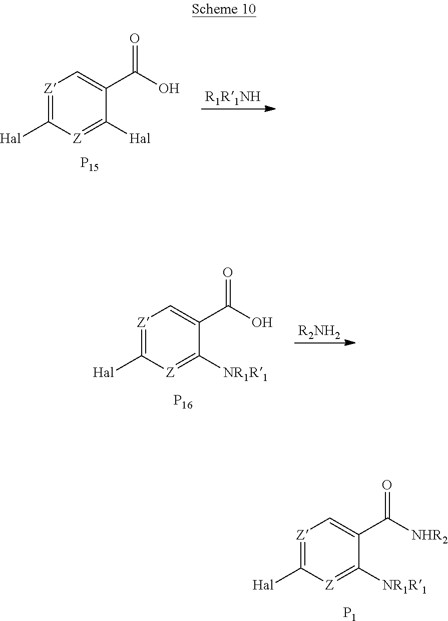

P₁₆ is obtained starting from the acid P₁₅ by monosubstitution with an amine of formula R₁R′₁ NH. In the case of an aliphatic or cycloaliphatic amine, the reaction can be carried out at ambient temperature and in a protic solvent such as an alcohol or water, or in an aprotic solvent such as THF (see also Ex.1.1.). In the case of an aniline, a strong base such as, for example, LiHMDS (((CH₃)₃Si)₂NLi) is added and the reaction is carried out under hot conditions (see also Ex.3.3.). The monosubstitution is described on pages 14-15 of FR 2917412 in the case where Z═N and Z′═CH, but can apply to other Z/Z′
Z═N, Z′═CH: P₁₅ is a 2,6-dihalonicotinic acid, for example 2,6-dichloronicotinic acid, which is commercially available;
Z═N, Z′═N: P₁₅ is a 2,4-dihalopyrimidine carboxylic acid, for example 2,4-dichloropyrimidine carboxylic acid, which is commercially available (CAS No. 37131-89-8);
Z═CH, Z′═CH: P₁₅ is a 2,4-dihalobenzoic acid, for example 2,4-dichlorobenzoic acid, which is commercially available (CAS No. 50-84-0).

In the case where Z and Z′ both represent N, and Hal represents a chlorine atom, P₁₆ may also be obtained starting from the commercially available compound ethyl 2,4-dichloropyrimidine-5-carboxylate:

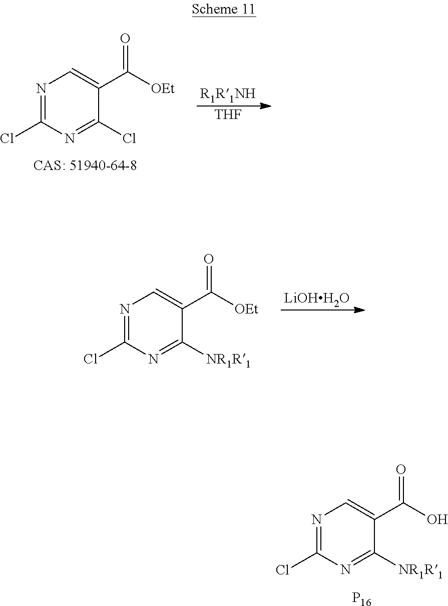

Scheme 11 using an ester function subsequently converted to an acid function also applies to the case where Z═N and Z′═CH: see the conditions in Chem. Pharm. Bull. 2000, 48(12), 1847-1853 (reactions in Tables 1 and 2).

P₁ is obtained starting from the acid in P₁₆ by amidation using the amine R₂NH₂ or a salt of this amine, for example the hydrochloride. The amidation can be carried out advantageously in the presence of an acid activator (also called coupling agent), for instance benzotriazol-1-yloxytris(dimethylamino)phosphonium hexafluorophosphate (or BOP, CAS No. 56602-33-6, see also Castro, B., Dormoy, J. R. Tetrahedron Letter 1975, 16, 1219). The reaction is preferably carried out in the presence of a base (such as triethylamine) at ambient temperature, in a solvent such as tetrahydrofuran (THF) or dimethylformamide (DMF). See Ex.1.2.

### Preparation of P₂

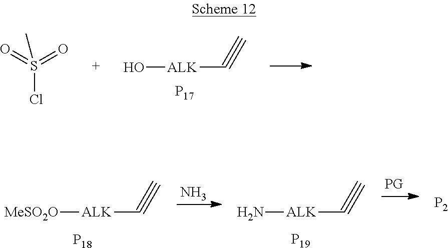

P₂ is obtained starting from P₁₇ according to Scheme 12 by conversion of the alcohol function to give an amine function via the intermediate P₁₈ carrying the mesyl leaving group, and then protection of P₁₉ with PG. Sodium azide can also be used in place of NH₃ so as to give an azide function, which is subsequently converted to give an amine function (see scheme II of Tetrahedron 1987, 43(21), 5145-58 and scheme 1 of Tetrahedron 2008, 64, 3578-3588). See prep.1.

### P₁₇ Compounds

P₁₇ may be either commercially available (for example, 3-butyn-1-ol CAS No. 927-74-2 or 2-propyn-1-ol CAS No. 107-19-7) or prepared according to the methods known to those skilled in the art.

P₄ Compounds

P₄ may be commercially available or prepared according to the methods known to those skilled in the art.

Hydrogenation of the cyano compound may, for example, be used, so as to obtain P₄ with n=1:

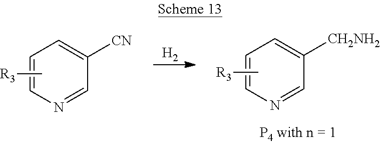

The hydrogenation conditions may be those described in Examples 19 and 20 of WO 00/46179 or in Synlett 2001, 10, 1623-1625.

The compounds 3-picolylamine (CAS No. 3731-52-0), 3-(2-aminoethyl)pyridine (CAS No. 20173-24-4), 2-amino-5-aminomethylpyridine (CAS No. 156973-09-0), 2-methyl-5-amino-methylpyridine (CAS No. 56622-54-9), 3-methyl-5-aminomethylpyridine (CAS No. 771574-45-9), 2-(BOC-amino)-5-aminomethylpyridine (CAS No. 187237-37-2) and 2,5-diaminopyridine (CAS No. 4318-76-7) are commercially available products. 2-Amino-5-aminomethylpyridine can also be prepared according to EP 0607804. 5-Aminomethyl-2-(dimethylamino)pyridine (CAS No. 354824-17-2) is commercially available or can be prepared according to Journal of Agricultural and Food Chemistry 2008, 56(1), 204-212. 2-Amino-3-methyl-5-aminomethyl-pyridine (CAS No. 187163-76-4) can be obtained by catalytic hydrogenation of the compound 6-amino-5-methylnicotinonitrile (CAS No. 183428-91-3), the amine function being doubly protected with BOC. Catalytic hydrogenation of 6-methylamino-3-pyridinecarbonitrile (CAS No. 261715-36-0) makes it possible to obtain 2-methylamino-5-aminomethylpyridine.

The preparation of 5-aminomethyl-2-(dimethylamino)pyridine (CAS No. 779324-37-7) and of 5-aminomethyl-2-(dimethylamino)pyridine (CAS No. 354824-17-2) in hydrochloride form is also described on page 106 of WO 2007/044449 (Examples 207 and 208).

### P₅ Compounds

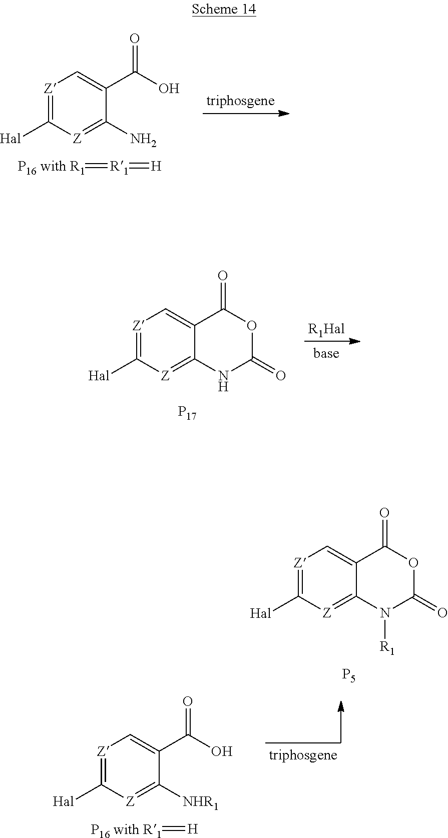

P₅ is obtained according to Scheme 14 starting from P₁₆ with R₁═R′₁═H or else starting from P₁₆ with R′₁═H. In the 1^{st} scenario, P₁₆ is reacted with triphosgene so as to obtain P₁₇. Next, P₁₇ is reacted with a halide R₁Hal in the presence of a strong base, for example NaH, in a polar solvent, for example DMF. The halide is more particularly an iodide (for example, ethyl iodide). In the 2^{nd} scenario, the triphosgene is reacted with P₁₆ where R′₁═H. The reaction with triphosgene can be carried out in dioxane at reflux for a long period of time (see Ex.2.1.).

### P₇ Compounds

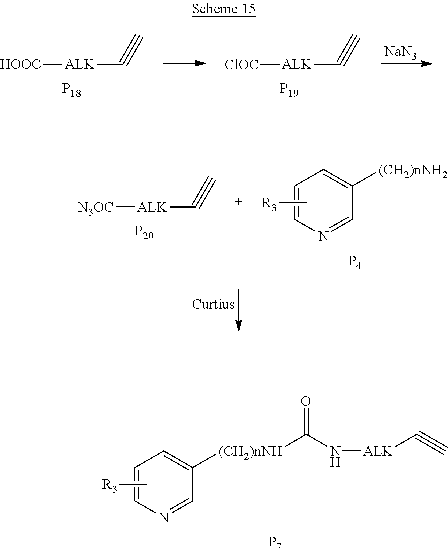

P₇ can be obtained by coupling between P₄ and P₁₉ in the presence of an agent for introducing the “C═O” unit. Another pathway for obtaining said compound is described in Scheme 15 and consists in reacting the acyl azide P₂₀ with the amine P₄ according to a Curtius rearrangement. The reaction conditions are illustrated by those of Ex.3.2.

### P₈ Compounds

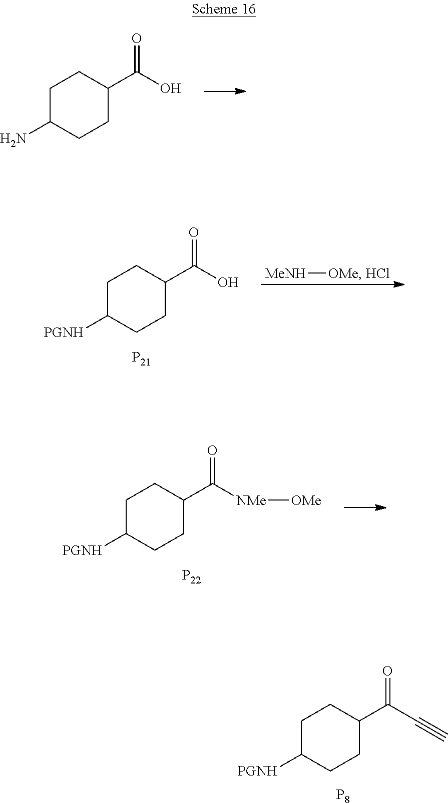

P₈ is obtained by reacting the Weinberg amide P₂₂ with an ethynyl magnesium halide HC≡CMgHal, for example the bromide. P₂₂ can be obtained starting from P₂₁ and from O,N-dimethylhydroxylamine according to the conditions described in detail in Synth. Comm. 2003, 33(23), 4013-4018 or in Synth. Comm. 2001, 31(13), 2011-2019 or else in the presence of an acid activator such as BOP, as described in detail in Ex.5.1. 4-Aminocyclohexanecarboxylic acid (cis or trans) is a commercially available product. PG represents more particularly BOC.

### P₉ Compounds

The acrylate P₉ is either commercially available or it can be obtained as indicated on page 945 of Helv. Chim. Acta 1973, 56(3), 944-958. The preparation of P₉ starting from imino ethers (the synthesis of which is itself described in JACS 1945, 67, 1017) is also described on page 8 of DE 2406198 or on page 11 of DE 2239815.

### P₁₂ Compound

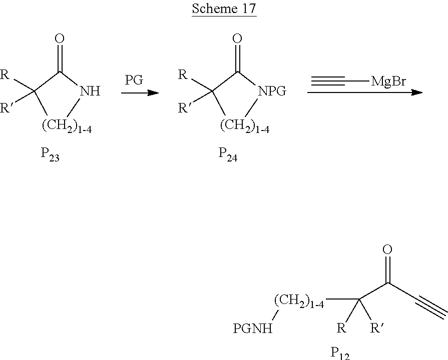

P₁₂ is obtained by reacting an ethynyl magnesium halide HC≡CMgHal, for example the bromide, with P₂₄. P₂₄ corresponds to compound P₂₃ which is protected on the NH function with a PG protective group such as BOC. P₂₃ may be a commercially available product, for instance 3,3-dimethyl-2-pyrrolidinone (CAS No. 4831-43-0), 3,3-dimethylpiperidinone (CAS No. 23789-83-5) or 5-azaspiro[2.4]heptan-4-one (CAS No. 3697-70-9). A method for obtaining P₂₃ with (CH₂)₃ is described in scheme 1 of J. Med. Chem. 1997, 40, 44-49. A method for obtaining P₂₃ with (CH₂)₂ is described in scheme 1 of J. Med. Chem. 1996, 39(9), 1898-1906. See also U.S. Pat. No. 5,776,959.

### R₂NH₃ Compounds

The R₂NH₂ amines are commercially available products or products already described in published documents:
1-(2-aminoethyl)piperidine: CAS No. 27578-60-5, described in Justus Liebigs Annalen der Chemie 1950, 566, 210-44, sold by Acros;
1-(2-aminoethyl)-4-piperidinol: CAS No. 129999-60-6, described in J. Med. Chem. 2005, 48(21), 6690-6695;
1-(2-aminoethyl)-3-piperidinol: CAS No. 847499-95-0, described in J. Med. Chem. 2005, 48(21), 6690-6695;
2-(4-methoxy-1-piperidinyl)ethylamine: CAS No. 911300-69-1, described in J. Med. Chem. 2007, 50(20), 4818-4831;
pyrrolidineethanamine: CAS No. 7154-73-6, described in Anales de Quimica 1974, 70(9-10), 733-737, sold by International Laboratory Ltd, 1067 Sneath Ln, San Bruno, Calif. 94066, USA;
azepan-1-ylethylamine: CAS No. 51388-00-2, described in Anales de Quimica 1974, 70(9-10), 733-737;
2-(1,1-dioxothiomorpholin-4-yl)ethylamine: CAS No. 89937-52-0, sold by Intern. Lab. Ltd;
N-(2-aminoethyl)thiamorpholine-1-oxide: CAS No. 1017791-77-3, sold by Sinova Inc. 3 Bethesda Metro Center, Suite 700, Bethesda, Md., 20814, USA.

A method for obtaining the compounds for which R₂ represents a (C₁-C₆)alkyl group substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, is described in Scheme 18 and is based on scheme 3 of Bioorg. Med. Chem. 2007, 15, 365-373 or on scheme 2 of Bioorg. Med. Chem. Lett. 2008, 18, 1378-1381:

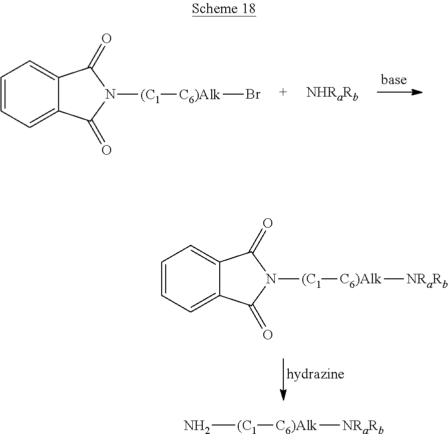

Another method described in Scheme 19 is based on FIG. 2 of Bioorg. Med. Chem. Lett. 2006, 16, 1938-1940:

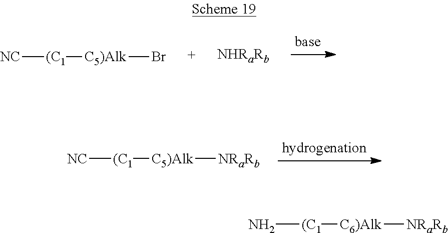

### Protection of the Primary or Secondary Amine Function

It may be necessary to use, in at least one of the stages, a protective group (PG) in order to protect one or more chemical function(s), in particular a primary or secondary amine function. For example, when Rₐ and R_{b} both represent a hydrogen atom, the amidation in Schemes 8, 9 or 10 is carried out using, for R₂NH₂, the compound ₂HN—(C₁-C₆)alkyl-NH-PG, where PG advantageously represents BOC (tert-butoxycarbonyl). Likewise, when the heterocycloalkyl group formed by Rₐ and R_{b} represents the piperazinyl group

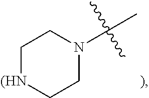

the —NH— function thereof can be advantageously protected using the following compound

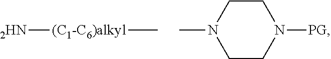

where PG advantageously represents BOC. Likewise, when R₃ represents the —NH₂ or —NHR_{c} group, the amine function can be advantageously protected with one or two PG group(s), preferably BOC. Use may, for example, be made of the following compound P:

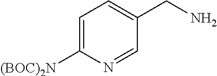

The chemical function(s) is (are) subsequently obtained by means of a (final or intermediate) deprotection stage, the conditions of which depend on the nature of the function(s) protected and on the protective group used. Reference may be made to “Protective groups in Organic Synthesis” by T. Greene, Wiley, 4^{th} ed., ISBN=978-0-471-69754-1, in particular to chapter 7 as regards amine-function-protecting groups. In the case of the protection of —NH₂ or —NH— functions with BOC, the deprotection stage is carried out in an acidic medium using, for example, HCl or trifluoroacetic acid. The associated salt (hydrochloride or trifluoroacetate) is thus, where appropriate, obtained.

### Obtaining of Salts

The salts are obtained during the deprotection stage described above or else by bringing the acid into contact with the compound in its base form.

In the above schemes, the starting compounds and the reactants, when the method for preparing them is not described, are commercially available or described in the literature, or else can be prepared according to methods which are described therein and which are known to those skilled in the art. Those skilled in the art may also use as a basis the operating conditions given in the examples which are described hereinafter.

### Obtaining of N-Oxides

The N-oxides of the compounds comprising an amine or a nitrogen atom are prepared according to the methods known to those skilled in the art, by reacting the amine with organic peracids such as peracetic acids, trifluoroperacetic acids, performic acids, perbenzoic acids or its derivatives such as 3-chloroperbenzoic acid, at temperatures of between 0° C. and 90° C., preferably at temperatures below 50° C.

According to a 3rd aspect, the invention concerns a pharmaceutical composition comprising a compound as defined above, in combination with a pharmaceutically acceptable excipient. The excipient is chosen from the usual excipients known to those skilled in the art, according to the pharmaceutical form and the method of administration desired. The method of administration may, for example, be oral or intravenous administration.

According to a 4th aspect, a subject of the invention is a medicament which comprises a compound as defined above, and also the use of a compound as defined above, for the manufacture of a medicament. It may be useful for treating a pathological condition, in particular cancer. The medicament (and also a compound according to the invention) may be administered in combination with one (or more) anticancer agent(s). This treatment may be administered simultaneously, separately or else sequentially. The treatment will be adapted by the practitioner according to the disease and the tumour to be treated.

According to a 5th aspect, the invention also concerns a method for treating the pathologies indicated above, which comprises the administration, to a patient, of an effective dose of a compound according to the invention or a pharmaceutically acceptable salt or hydrate or solvate thereof.

### EXAMPLES

The following examples illustrate the preparation of some compounds in accordance with the invention. The numbers of the compounds exemplified refer back to those given in the table hereinafter, which illustrates the chemical structures and the physical properties of some compounds according to the invention.

The compounds were analyzed by HPLC-UV-MS coupling (liquid chromatography, ultraviolet (UV) detection and mass detection). The apparatus used is composed of an Agilent chromatography system equipped with an Agilent diode array detector and with a Waters ZQ single quadripole mass spectrometer or a Waters Quattro-Micro triple quadripole mass spectrometer.

### Mass Spectrometry Conditions

The liquid chromatography/mass spectrometry (LC/MS) spectra were recorded in positive electrospray (ESI) mode, in order to observe the ions resulting from the protonation of compounds analyzed (MH⁺) or from the formation of adducts with other cations, such as Na⁺, K⁺, etc. The ionization parameters are as follows: cone voltage: 20 V; capillary voltage: 3 kV; source temperature: 120° C.; desolvation temperature: 450° C.; desolvation gas: N₂ at 450 l/h.

The HPLC conditions are chosen from one of the following methods:

| Con- | | | |
|---|---|---|---|
| ditions | TFA15 | TFA3 | AcONH₄ 15 min |
| Column | Symmetry C18 | Acquity BEH C18 | XTerra MS C18 |
| | (50 × 2.1 mm; 3.5 μm) | (50 × 2.1 mm; 1.7 μm) | (50 × 2.1 mm; 3.5 μm) |
| Eluent A | H₂O + TFA 0.005% at | H₂O + TFA 0.05% at | 10 mM AcONH₄ at pH ~7/ |
| | approximately pH 3.1 | approximately pH 3.1/CH₃CN | CH₃CN (97/3) |
| | | (97/3) | |
| Eluent B | CH₃CN + TFA 0.005% | CH₃CN + TFA 0.035% | CH₃CN |
| Gradient | 100:0 (0 min) [symbol] 10:90 (10 | 100:0 or 99:1 (0 min) [symbol] 5:95 (2.3 | 100:0 (0 min) [symbol] 10:90 (10 min) |
| A:B | min) [symbol] 10:90 (15 min) [symbol] | min) [symbol] 5:95 (2.9 min) [symbol] 100:0 or | [symbol] 10:90 (15 min) [symbol] 100:0 (16 |
| | 100:0 (16 min) [symbol] 100:0 | 99:1 (3 min) [symbol] 100:0 or 99:1 (3.5 | min) [symbol] 100:0 (20 min) |
| | (20 min) | min) | |
| T_{column} | 30° C. | 40° C. | 30° C. |
| Flow rate | 0.4 ml/min | 1 ml/min | 0.4 ml/min |
| Detection | λ = 220 nm | λ = 220 nm | λ = 220 nm |

TFA: trifluoroacetic acid

### NMR Conditions

The ¹H NMR spectra are recorded on a Bruker Avance 250/Bruker Avance 400 or Bruker Avance II 500 spectrometer. The central peak of DMSO-d6 (2.50 ppm) is used as an internal reference. The following abbreviations are used: s: singlet; d: doublet; dd: double of doublets; t: triplet; q: quadruplet; m: unresolved peak/multiplet; br.s: broad signal.

### Preparation 1

### Prep.1.1.

### Methanesulphonic acid but-3-ynyl ester

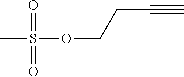

In a round-bottomed flask, 10 ml (132.12 mmol) of but-3-yn-1-ol and 28 ml (201.16 mmol) of triethylamine are diluted in 500 ml of CH₂Cl₂ (DCM), the mixture is cooled to 0° C. and then 11.3 ml (145.4 mM) of methanesulphonyl chloride are added. The mixture is stirred for 4 h while keeping the temperature at 0° C. The organic phase is washed with water, a 1N solution of HCl, a saturated solution of Na₂HCO₃, and then a saturated solution of NaCl. The resulting product is dried over Na₂SO₄, filtered and evaporated. 19.15 g (yield=97%) of a translucent oil are obtained.

### Prep.1.2

### But-3-ynylcarbamic acid tert-butyl ester

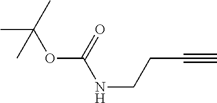

In a round-bottomed flask, 20 g (134.97 mM) of methanesulphonic acid but-3-ynyl ester are diluted in 100 ml of EtOH. 200 ml (3344.49 mM) of an aqueous solution of ammonia at 32% are added. The mixture is heated at 50° C. for 3 h. The mixture is brought back to ambient temperature (AT) and then 32.4 g (148.47 mM) of Boc₂O dissolved beforehand in 250 ml of acetonitrile are added. The mixture is stirred for 72 h at AT. The mixture is filtered and then the filtrate is evaporated. The residue is taken up in DCM, the organic phase is washed with a saturated solution of NaCl, the resulting product is separated by settling out, and the organic phase is dried over Na₂SO₄. The solvents are evaporated to dryness. The resulting product is purified by flash chromatography with 100% DCM. 17.2 g (yield=55.2%) of a translucent oil are obtained.

### Preparation 2

### Prep.2.1.

### Pyridin-3-ylmethylcarbamic acid tert-butyl ester

3.24 g (30 mM) of pyridin-3-ylmethylamine are dissolved in 100 ml of DCM; 7.20 g (33 mM) of BOC₂O and 4.59 ml (33 mM) of triethylamine are added. The mixture is stirred at ambient temperature for 3 h. The solvents are evaporated off and the residue is taken up with DCM. The resulting product is washed with water and then a saturated solution of NaCl. The resulting product is dried over sodium sulphate, and filtered, and then the filtrate is evaporated off. The residue is purified by flash chromatography (99/1 DCM/CH₃OH-95/5 DCM/CH₃OH). 5.64 g (90%) are obtained.

LCMS (NEUTRAL) MH⁺=209; tr=5.99.

### Prep.2.2.

### (1-Oxypyridin-3-ylmethyl)carbamic acid ten-butyl ester

5.64 g (27.08 mM) of pyridin-3-ylmethylcarbamic acid tert-butyl ester are dissolved in 40 ml of methanol; 4.55 g (54.1 mM) of sodium bicarbonate and then 50 ml of water are added, and 12.24 g (19.9 mM) of potassium peroxymonosulphate, solubilized beforehand in 50 ml of water, are added dropwise. The mixture is stirred at ambient temperature for 18 h. It is diluted with 100 ml of DCM, filtered and separated by settling out. The aqueous phase is extracted with DCM, and washing is carried out with a saturated solution of NaCl. The resulting product is dried over sodium sulphate and filtered, and then the filtrate is evaporated off. 5.74 g (95%) are obtained.

LCMS (NEUTRAL) MH⁺=225; tr=5.72.

### Prep.2.3.

### (1-Oxypyridin-3-yl)methylamine dihydrochloride

5 ml (20 mM) of 4M HCl in dioxane are cooled to 0° C. 1.49 g (6.64 mM) of (1-oxypyridin-3-ylmethyl)carbamic acid tert-butyl ester are added. The mixture is brought back to AT and then stirred for 2 h. 10 ml (20 mM) of 4M HCl in dioxane are added and the mixture is stirred for a further 2 h. The resulting product is evaporated to dryness. 1.3 g (100%) are obtained.

LCMS (NEUTRAL) MH⁺=125.

### Example 1

### 6-{4-[3-(6-Aminopyridin-3-ylmethyl)ureido]butyl}-2-ethylamino-N-methylnicotinamide (compound No. 5; illustrates scheme 1)

### 1.1. 6-chloro-2-ethylaminonicotinic acid

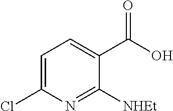

In a round-bottomed flask, 26.1 g (0.136 M) of 2,6-dichloronicotinic acid are mixed with 180 ml of an aqueous solution of ethylamine at 70%. The mixture is stirred at AT for 5 d. The mixture is evaporated under reduced pressure (RP). The residue is taken up with 100 ml of water. The resulting product is cooled using an ice bath and acidified to pH 3 with a 5N HCl solution. The precipitate is filtered off, washed with cold water and vacuum-dried over P₂O₅ at 60° C. 24.93 g (91.4%) of a white solid are obtained. Mp=157-159° C.

### 1.2. 6-Chloro-2-methylaminonicotinamide

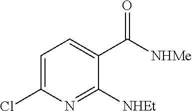

In a round-bottomed flask, 5.0 g (24.92 mM) of 6-chloro-2-ethylaminonicotinic acid are dissolved in 300 ml of THF. 10.41 ml (74.77 mM) of triethylamine are added, followed by 14.95 ml (29.91 mM) of a 2N solution of methylamine in THF and subsequently 13.22 g (29.91 mM) of BOP. The mixture is stirred at AT for 15 h. The solvent is evaporated off and the residue is taken up with ethyl acetate. The organic phase is washed with water and then a saturated solution of NaCl. The resulting product is dried over Na₂SO₄, filtered and evaporated. The residue is purified by flash chromatography (gradient DCM-MeOH 1 to 10%). 4.1 g are obtained (yield: 77%) (LCMS (TFA3) tr=1.19 min).

### 1.3. [4-(6-Ethylamino-5-methylcarbamoylpyridin-2-yl)but-3-ynyl]carbamic acid tert-butyl ester

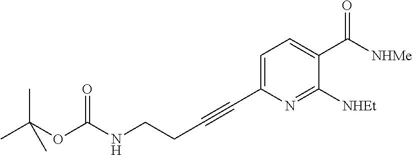

2.9 g (3 mM) of 6-chloro-2-methylaminonicotinamide are dissolved in 20 ml of DMF. 2.29 g (13.57 mM) of tert-butyl but-3-yn-1-ylcarbamate and 6.61 ml (47.50 mmol) of triethylamine are added. The mixture is degassed with argon for 30 min and then 0.47 g (0.68 mM) of dichlorobis(triphenylphosphine)palladium(II) and 0.13 g (0.068 mM) of CuI are added. The mixture is stirred while heating at 90° C. for 12 h. The mixture is evaporated and the residue is taken up with DCM; the resulting product is washed with water and dried over sodium sulphate; the resulting product is filtered and evaporated. The residue is purified by flash chromatography with 100 DCM/90-10 DCM-MeOH. 2.5 g are obtained (yield=45%) (LCMS (TFA3): tr=2.15).

### 1.4. 6-(4-Aminobut-1-ynyl)-2-ethylamino-N-methylnicotinamide in trifluoroacetate form

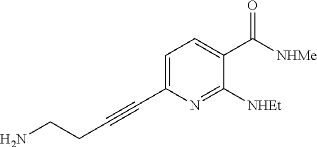

2.5 g (7.22 mM) of [4-(6-ethylamino-5-methylcarbamoylpyridin-2-yl)but-3-ynyl]carbamic acid tert-butyl ester are dissolved in 30 ml of DCM. The mixture is cooled using an ice bath and 11.12 ml of TFA are added. The mixture is stirred at AT for 15 h. The solvent is evaporated off. The residue is purified by flash chromatography with 100 DCM/80-20 DCM-MeOH. 0.8 g is obtained (yield=45%) (LCMS (TFA3): tr=1.62 min).

### 1.5. 6-(4-Aminobutyl)-2-ethylamino-N-methylnicotinamide in trifluoroacetate form

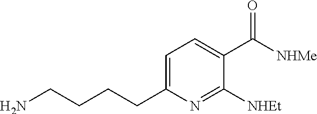

0.8 g (3.25 mM) of 6-(4-aminobut-1-ynyl)-2-ethylamino-N-methylnicotinamide is dissolved in 50 ml of ethanol and the mixture is hydrogenated at AT and under normal pressure in the presence of 0.07 g (0.06 mM) of Pd/C at 10%. The resulting product is filtered through Whatman paper and the filtrate is evaporated off. 0.76 g is obtained (yield=93.8%) (LCMS (TFA3): tr=1.52 min)

### 1.6. 6-{4-[3-(6-Aminopyridin-3-ylmethyl)ureido]butyl}-2-ethylamino-N-methylnicotinamide

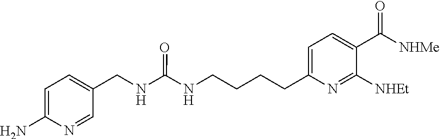

0.3 g (1.20 mM) of 6-(4-aminobutyl)-2-ethylamino-N-methylnicotinamide is dissolved in 30 ml of THF; 0.50 ml (3.6 mM) of triethylamine, 0.175 g (1.44 mM) of DMAP and 0.368 g (1.44 mM) of DSC are successively added. The mixture is stirred at AT for 4 h. 0.465 g (1.44 mM) of di-tert-butyl[5-(aminomethyl)pyridin-2-yl]imidodicarbonate is subsequently added and the mixture is stirred at AT for 12 h. The solvents are evaporated off and the residue is taken up with DCM; the resulting product is washed with water and brine. The resulting product is separated by settling out and the organic phase is dried over Na₂SO₄. Purification is carried out by flash chromatography with 99-1/80-20 DCM-MeOH. The residue is dissolved in 20 ml of DCM; the resulting product is cooled using an ice bath and 30 eq of TFA are added. The mixture is stirred at AT for 12 h. The mixture is evaporated and the residue is taken up with a 10% Na₂CO₃ solution. The precipitate is filtered off and washed with water. The resulting product is vacuum-dried over P₂O₅ at 60° C. 0.33 g of the product is obtained (yield=70%). LCMS (TFA3) tr=0.53 min; ¹H NMR (250 MHz, DMSO-d6) 1.14 (t, 3H), 1.27-1.49 (m, 2H), 1.52-1.75 (m, 2H), 2.51-2.60 (m, 2H), 2.72 (d, 3H), 3.01 (q, 2H), 3.34-3.48 (m, 2H), 3.97 (d, 2H), 5.75 (s, 2H), 5.83 (t, 1H), 6.05 (t, 1H), 6.39 (d, 2H), 7.26 (dd, 1H), 7.70-7.85 (m, 2H), 8.20-8.41 (m, 2H).

### Example 2

### 6-[4-({[(6-Aminopyridin-3-yl)methyl]carbamoyl}amino)butyl]-N-[2-(1,1-dioxidothiomorpholin-4-yl)ethyl]-2-(ethylamino)nicotinamide (compound No. 19; illustrates scheme 2)

### 2.1. 7-chloro-1-ethyl-2H-pyrido[2,3-d][1,3]oxazine-2,4(1H)-dione

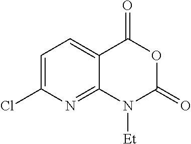

2 g (10 mM) of 6-chloro-2-(ethylamino)nicotinic acid are dissolved in 25 ml of dioxane and then 1.48 g (5 mM) of triphosgene are added. The mixture is brought to reflux for 48 h. After a return to ambient temperature (AT), the mixture is filtered and the precipitate is washed with water and vacuum-dried over P₂O₅. 1.70 g are obtained. Yield=75.2%.

### 2.2. tert-Butyl {4-[5-{[2-(1,1-dioxidothiomorpholin-4-yl)ethyl]carbamoyl}-6-(ethylamino)pyridin-2-yl]but-3-yn-1-yl}carbamate

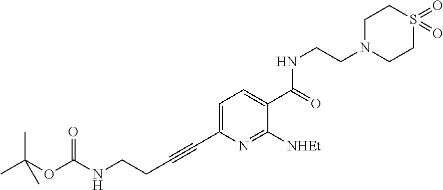

0.680 g (3 mM) of the compound obtained in stage 1 is dissolved in 20 ml of DMF. 1.05 g (6 mM) of tert-butyl but-3-yn-1-ylcarbamate and 3 ml of triethylamine are added. The mixture is degassed with argon for 30 min, and then 0.105 g (0.15 mM) of dichlorobis(triphenylphosphine)palladium(II) and 0.04 g (0.21 mM) of CuI are added. The mixture is stirred at AT for 16 h. 1.07 g (6 mM) of 2-(1,1-dioxidothiomorpholin-4-yl)ethanamine in 3 ml of DMF are added and the mixture is stirred at AT for 3 h. The mixture is evaporated and the residue is taken up with DCM; the resulting product is washed with water and dried over sodium sulphate; the resulting product is filtered and evaporated. The residue is purified by flash chromatography with 100 DCM/95-5 DCM-MeOH. 0.91 g is obtained (yield=61%) LCMS (TFA3): tr=2.06 min.

### 2.3. tert-Butyl {4-[5-{[2-(1,1-dioxidothiomorpholin-4-yl)ethyl]carbamoyl}-6-(ethylamino)pyridin-2-yl]butyl}carbamate

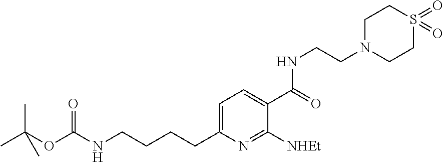

0.9 g (1.82 mM) of compound obtained in stage 2.2 is dissolved in 40 ml of ethanol and the mixture is hydrogenated at AT and under normal pressure in the presence of 0.04 g of Pd/C at 10%. The resulting product is filtered through Whatman paper and the filtrate is evaporated off. Purification is carried out by flash chromatography with 100 DCM/95-5 DCM-MeOH. 0.77 g is obtained (yield=85%) (LCMS (TFA3): tr=1.95 min).

### 2.4. 6-(4-Aminobutyl)-N-[2-(1,1-dioxidothiomorpholin-4-yl)ethyl]-2-(ethylamino)nicotinamide di(trifluoroacetate)

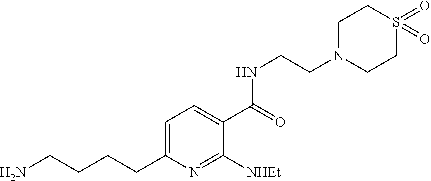

0.754 g (1.52 mM) of compound obtained in stage 2.3 is dissolved in 30 ml of DCM. The mixture is cooled using an ice bath and 2.57 ml of TFA are added. The mixture is stirred at AT for 18 h. The resulting product is evaporated; the residue is taken up with Et₂O and the resulting product is again evaporated. 0.945 g is obtained (yield=99.7%) (LCMS (TFA3): tr=1.56 min).

### 2.5. tert-Butyl (5-{[({4-[5-{[2-(1,1-dioxidothiomorpholin-4-yl)ethyl]carbamoyl}-6-(ethylamino)pyridin-2-yl]butyl}carbamoyl)amino]methyl}pyridin-2-yl)carbamate

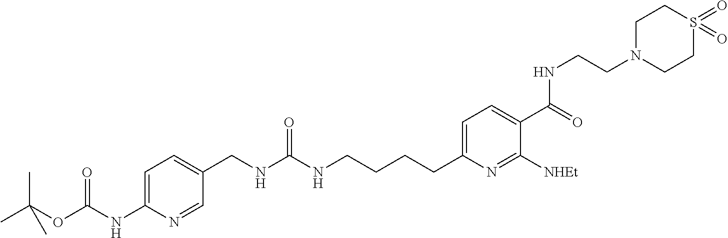

0.944 g (1.51 mM) of compound obtained in stage 2.4 is dissolved in 50 ml of THF; 0.84 ml (6.04 mM) of triethylamine, 0.277 g (2.26 mM) of DMAP and 0.582 g (2.26 mM) of DSC are successively added. The mixture is stirred at AT for 2 h. 25 ml of DMF and 0.405 g (1.81 mM) of tert-butyl[5-(aminomethyl)pyridin-2-yl]carbamate are subsequently added and the mixture is stirred at AT for 18 h. The solvents are evaporated off and the residue is taken up with DCM; the resulting product is washed with water and an insoluble material is filtered off. The resulting product is separated by settling out and the organic phase is dried over Na₂SO₄. Purification is carried out by flash chromatography with 95-5/90-10 DCM-MeOH. 0.54 g is obtained (yield=55.2%) (LCMS (TFA3): tr=1.87 min).

### 2.6. 6-[4-({[(6-Aminopyridin-3-yl)methyl]carbamoyl}amino)butyl]-N-[2-(1,1-dioxidothiomorpholin-4-yl)ethyl]-2-(ethylamino)nicotinamide

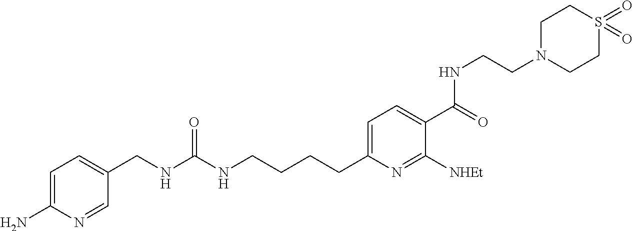

0.513 g of compound obtained in stage 5 is dissolved in 20 ml of DCM; the mixture is cooled using an ice bath and 1.34 ml (17.45 mM) of TFA are added. The mixture is stirred at AT for 18 h. The mixture is evaporated and the residue taken up with a 10% Na₂CO₃ solution. The precipitate is filtered off and washed with water. The resulting product is vacuum-dried over P₂O₅ at 60° C. 0.37 g is obtained (yield=85.2%); LCMS (TFA3) tr=0.53 min; ¹H NMR (250 MHz, DMSO-d6) 1.14 (t, 3H), 1.30-1.49 (m, 2H), 1.52-1.78 (m, 2H), 2.56 (t, 2H), 2.64 (t, 2H), 2.88-3.17 (m, 10H), 3.22-3.50 (m, 4H), 3.98 (d, 2H), 5.76 (s, 2H), 5.82 (t, 1H), 6.05 (t, 1H), 6.34-6.46 (m, 2H), 7.27 (dd, 1H), 7.73-7.89 (m, 2H), 8.19-8.37 (m, 2H).

### Example 3

### N-Methyl-2-phenylamino-6-[4-(3-pyridin-3-ylmethylureido)butyl]-nicotinamide (compounds No. 3 and No. 10; illustrates the schemes 3 and 6)

### 3.1. Pent-4-ynoyl chloride

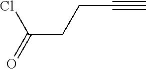

10 g (101.94 mM) of 4-pentynoic acid are dissolved in 100 ml of DCM, and 0.4 ml (5.2 mM) of DMF is added, followed by 11 ml (126.1 mM) of oxalyl chloride, dropwise. The mixture is stirred at AT for 2 h. The mixture is evaporated to dryness and then distilled under RP (water-jet pump) (40±2° C.). 10.4 g of a translucent oil are obtained (yield=87%).

### 3.2. 1-But-3-ynyl-3-pyridin-3-ylmethylurea

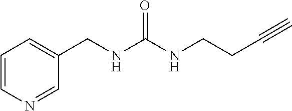

9.1 g (78.77 mM) of 4-pentynoic acid chloride are diluted in 130 ml of acetonitrile. 5.6 g (86.64 mM) of NaN₃ are added and then the mixture is heated at 70° C. for 2 h. 9.3 g (86.64 mM) of 3-(aminomethyl)pyridine, solubilized beforehand in 15 ml of acetonitrile, are added and then the mixture is heated at 70° C. for 3 h. The solvents are evaporated off, the residue is taken up in DCM, and the resulting product is washed successively with H₂O and brine. The resulting product is dried over Na₂SO₄, filtered and evaporated. The residue is purified by flash chromatography with 100% DCM to 90/10 DCM/MeOH. 7.11 g of a white powder are obtained (yield=44%). LCMS (TFA15): tr=4.1 min.

### 3.3. 6-Chloro-2-phenylaminonicotinic acid

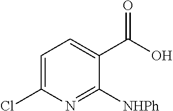

1.52 g (16.4 mM) of aniline are dissolved in 25 ml of THF, and 25 ml (25 mM) of a 1N solution of LiHMDS in THF are added dropwise. The reaction medium is stirred at 75° C. for 1 h. 1.5 g (7.8 mM) of dichloronicotinic acid dissolved beforehand in 25 ml of THF are added dropwise. The mixture is stirred at AT for 15 h. The mixture is diluted with water and then acidified to pH=2 with 5N HCl. The mixture is extracted with ethyl acetate, and the organic phase is washed successively with H₂O and brine. The organic phase is dried with Na₂SO₄, filtered, and then evaporated to dryness. The residue is purified by flash chromatography with 99/1 DCM/MeOH to 90/10 DCM/MeOH. 1.17 g of a white solid are obtained (yield=61 mol %). (LCMS (TFA15): tr=9.5 min).

### 3.4. 6-Chloro-N-methyl-2-phenylaminonicotinamide

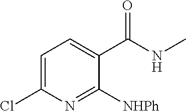

In a round-bottomed flask, 0.5 g (2.01 mM) of 6-chloro-2-phenylaminonicotinic acid is dissolved in 20 ml of THF. 0.84 ml (6.03 mM) of triethylamine is added, followed by 2.01 ml (4.02 mM) of a 2N solution of methylamine in THF and subsequently 0.89 g (2.01 mM) of BOP. The mixture is stirred at AT for 15 h. The solvent is evaporated off and the residue is taken up with ethyl acetate. The organic phase is washed with water and then a saturated solution of NaCl. It is dried over Na₂SO₄, filtered and evaporated. The residue is purified by flash chromatography (100% DCM isocratic gradient). 0.5 g of a white solid is obtained (yield: 96%) (LCMS (TFA15): tr=9.27 min).

### 3.5. N-Methyl-2-phenylamino-6-[4-(3-pyridin-3-ylmethylureido)but-1-ynyl]nicotinamide

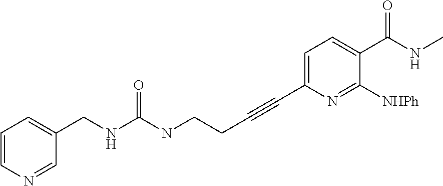

0.5 g (1.91 mM) of 6-chloro-2-phenylamino-N-methylnicotinamide is dissolved in 15 ml of DMF. 0.38 g (1.91 mM) of 1-but-3-ynyl-3-pyridin-3-ylmethylurea and 0.93 ml (6.69 mM) of triethylamine are added. The mixture is degassed with argon for 30 min and then 0.06 g (0.10 mM) of dichlorobis(triphenylphosphine)palladium(II) and 0.02 g (0.10 mM) of CuI are added. The mixture is stirred while heating at 90° C. for 12 h. The resulting product is evaporated and the residue is taken up with DCM; the resulting product is washed with water and dried over sodium sulphate; the resulting product is filtered and evaporated. The residue is purified by flash chromatography with 100 DCM/90-10 DCM-MeOH. 0.4 g is obtained (yield=49%) LCMS (TFA15): tr=5.96 min; ¹H NMR (400 MHz, DMSO-d6) 2.60 (t, 2H), 2.81 (d, 3H), 3.23-3.31 (m, 2H), 4.24 (d, 2H), 6.26 (t, 1H), 6.60 (t, 1H), 6.94 (d, 1H), 6.98 (t, 1H), 7.27-7.38 (m, 3H), 7.59-7.71 (m, 3H), 8.03 (d, 1H), 8.46 (br. s., 2H), 8.66-8.83 (m, 1 H), 11.03 (s, 1H).

### 3.6. N-Methyl-2-phenylamino-6-[4-(3-pyridin-3-ylmethylureido)butyl]nicotinamide

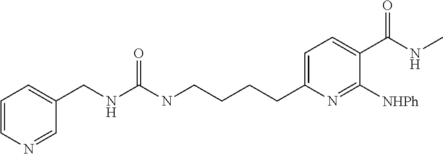

0.27 g (0.63 mM) of N-methyl-2-phenylamino-6-[4-(3-pyridin-3-ylmethylureido)but-1-ynyl]nicotinamide is dissolved in 30 ml of ethanol and the mixture is hydrogenated at AT under normal pressure in the presence of 0.013 g (0.01 mM) of Pd/C at 10%. The resulting product is filtered through Whatman paper and the filtrate is evaporated off. The compound is recrystallized from a minimum amount of ethyl acetate, and the resulting product is filtered and dried. 0.15 g of a white powder is obtained (yield=57%). LCMS (TFA15) tr=5.82 min; ¹H NMR (250 MHz, DMSO-d6) 1.38 (qd, 2H), 1.51-1.82 (m, 2H), 2.63 (t, 2H), 2.75 (d, 3 H), 3.00 (q, 2H), 4.16 (d, 2H), 5.96 (t, 1H), 6.31 (t, 1H), 6.66 (d, 1H), 6.89 (t, 1H), 7.17-7.36 (m, 3H), 7.61 (d, 1H), 7.69 (d, 2H), 7.95 (d, 1H), 8.42 (d, 2H), 8.60 (d, 1H), 11.04 (s, 1H).

### Example 4

### 6-(4-(3-((6-Aminopyridin-3-yl)methyl)ureido)-2-methylbutan-2-yl)-N-methyl-2-(phenylamino)nicotinamide (compound No. 34; illustrates scheme 9)

### 4.1. tert-Butyl 3,3-dimethyl-2-oxopyrrolidine-1-carboxylate

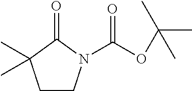

15.3 ml of diisopropylamine are added, at −70° C., to a solution of 44 ml of 2.5 M nBuLi in hexane. The medium is heated at −10° C. for 5 min and then cooled to −70° C., and 10 g of tert-butyl 2-oxopyrrolidine-1-carboxylate are added dropwise in solution in 100 ml of THF. The medium is stirred at −70° C. for 1 h 30. 14 ml of methyl iodide are added and the mixture is stirred for 2 h at −70° C. The medium is allowed to come back up to −50° C., and a white solid precipitates. 830 mg of tBuOK are added and the medium is left to come back up to AT overnight. The medium is hydrolyzed, extracted in EtOAc, and the organic phases are washed with water and with brine, dried over MgSO₄, and then evaporated to give 11 g of a brown oil, which is a mixture of the monoalkyl compound and the dialkyl compound. 8 ml of diisopropylamine are added, at −70° C., to the solution of 22 ml of 2.5 M BuLi in hexane. The medium is heated at −10° C. for 5 min and then cooled to −70° C., and the crude product from the preceding stage, in solution in 100 ml of THF, is added. The medium is stirred at −70° C. for 1 h 30. 7 ml of methyl iodide are added and the mixture is stirred for 2 h at −70° C. The medium is allowed to come back up to AT overnight. The medium is hydrolyzed, extracted in EtOAc, and the organic phases are washed with water and with brine, dried over MgSO₄, and then evaporated to give 10 g of a brown oil. The crude product is chromatographed on silica, eluent: gradient: cyclohexane to 50/50 cyclohexane/EtOAc, so as to give 7 g of the expected product.

### 4.2. tert-Butyl 3,3-dimethyl-4-oxohex-5-ynylcarbamate

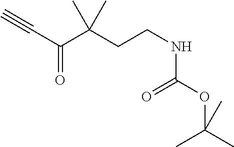

900 mg of the compound obtained in the preceding stage are dissolved in 10 ml of THF. After cooling to −70° C., 25 ml of ethynyl magnesium bromide in solution at 0.5 M in THF are added dropwise. The medium is stirred at −45° C. for 30 min and then the temperature is allowed to come back up to 18° C. The medium is hydrolyzed, extracted in EtOAc, and the organic phases are washed with water and with brine, dried over MgSO₄, and then evaporated to give 974 mg of a yellow oil.

### 4.3. Ethyl 6-(4-(tert-butoxycarbonylamino)-2-methylbutan-2-O-2-(phenylamino)nicotinate

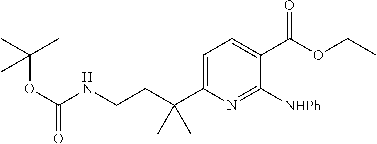

840 mg of ethyl 3-amino-3-(phenylamino)acrylate are added to a solution of 0.974 g of the compound obtained in the preceding stage, in 30 ml of ethanol. The mixture is refluxed for 3 days. After returning to AT, the medium is filtered. The filtrate is evaporated to give 1.65 g of the expected product.

### 4.4. Ethyl 6-(4-amino-2-methylbutan-2-yl)-2-(phenylamino)nicotinate

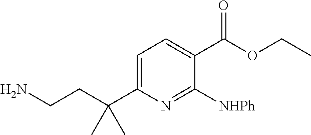

9.6 ml of a 4M solution of HCl in dioxane are added to a solution of 1.65 g of the compound obtained in the preceding stage, in 5 ml of DCM. After stirring at AT for 12 h, the medium is concentrated and then taken up in DCM. The organic phase is washed with a saturated solution of NaHCO₃ and then with brine, and dried over MgSO₄, and then evaporated. 1.25 g of the expected product are obtained.

### 4.5. Ethyl 6-(4-(3-((6-(bis(tert-butoxycarbonyl)amino)pyridin-3-yl)methyl)ureido)-2-methylbutan-2-yl)-2-(phenylamino)nicotinate

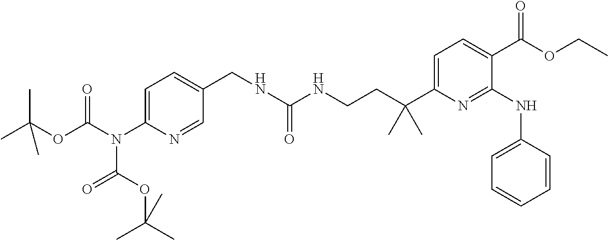

A solution of 1.07 g of the compound obtained in the preceding stage, in 25 ml of THF, are added, dropwise, to a solution of 500 mg of DMAP, 0.54 ml of triethylamine and 1 g of DSC in 20 ml of THF. After stirring at AT for 5 min, 1.27 g of bis-tert-butyl 5-(aminomethyl)pyridin-2-ylcarbamate are added. After 12 h at AT, the medium is concentrated and then taken up in DCM. The organic phase is washed with a saturated solution of NaHCO₃ then with brine, then dried over MgSO₄, and then evaporated. 3.89 g of a crude product are obtained, which product is purified by silica gel chromatography to give 2 g of the desired compound.

### 4.6. 6-(4-(3-((6-(tert-Butoxycarbonylamino)pyridin-3-yl)methyl)ureido)-2-methylbutan-2-yl)-2-(phenylamino)nicotinic acid

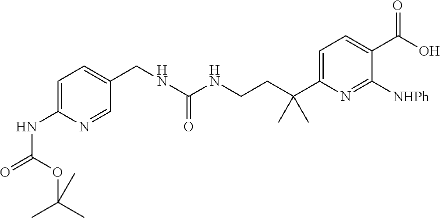

2 ml of 5M NaOH are added to a solution of 2 g of the compound obtained in the preceding stage in 10 ml of ethanol. The medium is stirred at AT for 12 h and then concentrated and taken up in a mixture of water and ethyl ether. After separation by settling out, the aqueous phase is acidified to pH=4 with 1N HCl, and then extracted in DCM. The combined organic phases are dried over MgSO₄ and then evaporated so as to give 1 g of the expected product (a Boc group having been cleaved).

### 4.7. tert-Butyl 5-((3-(3-methyl-3-(5-(methylcarbamoyl)-6-(phenylamino)pyridin-2-yl)butyl)ureido)methyl)pyridin-2-ylcarbamate

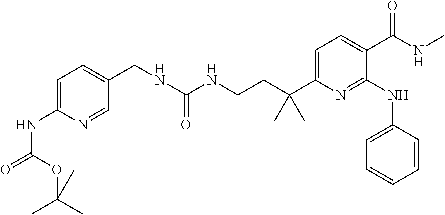

1 g of the compound obtained in the preceding stage is mixed with 123 mg of methylamine hydrochloride, 172 mg of HOBt, 0.95 ml of DIPEA and 1.17 g of TBTU in 5 ml of DCM. The medium is stirred at AT for 2 h. The medium is hydrolyzed, diluted in DCM, washed with water and with brine, then dried over MgSO₄ and then evaporated so as to give 0.9 g of a brown oil. The crude product is chromatographed on silica gel (gradient: DCM to 95/5 DCM/MeOH) so as to give 284 mg of the expected compound.

### 4.8. 6-(4-(3-((6-Aminopyridin-3-yl)methyl)ureido)-2-methylbutan-2-yl)-N-methyl-2-(phenylamino)nicotinamide

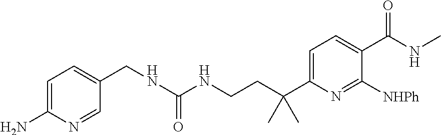

284 mg of the compound obtained in the preceding stage are dissolved in 0.5 ml of DCM, and 1 ml of TFA is added. The medium is stirred at AT for 2 h. Water and then a saturated solution of NaHCO₃ are added and the mixture is extracted in DCM. The organic phases are washed with water and with brine and then dried over MgSO₄ and evaporated. The crude product is purified by silica chromatography so as to give 146 mg of a white powder. Mp=134° C.; ¹H NMR (400 MHz, DMSO-d6) 1.31 (s, 6H), 1.75-1.86 (m, 2H), 2.77-2.88 (m, 5 H), 3.94 (d, 2H), 5.68 (t, 1H), 5.74 (s, 2H), 6.01 (t, 1H), 6.36 (d, 1H), 6.87 (d, 1H), 6.94 (t, 1 H), 7.23 (dd, 1H), 7.30 (t, 2H), 7.72 (d, 2H), 7.75 (d, 1H), 8.03 (d, 1H), 8.66 (q, 1H), 11.05 (s, 1H).

### Example 5

### 6-((1s,4s)-4-(3-((6-Aminopyridin-3-yl)methyl)ureido)cyclohexyl)-2-(phenylamino)-N-(2-(piperidin-1-yl)ethyl)nicotinamide (compound No. 17; illustrates scheme 8)

### 5.1. tert-Butyl (1s,4s)-4-(methoxy(methyl)carbamoyl)cyclohexylcarbamate

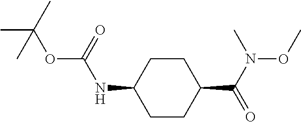

3 g of (1s,4s)-4-(tert-butoxycarbonylamino)cyclohexanecarboxylic acid are mixed with 1.22 g of O,N-dimethylhydroxylamine hydrochloride, 5.2 ml of triethylamine and 5.54 g of BOP in 125 ml of THF. The medium is stirred at AT for 2 days. The medium is filtered and then concentrated. The crude product is chromatographed on silica gel (gradient: DCM to 98/2 DCM/MeOH) so as to give 2.33 g of the expected compound. MH⁺=309 at tr=2.3 min.

### 5.2. tert-Butyl (1s,4s)-4-propioloylcyclohexylcarbamate

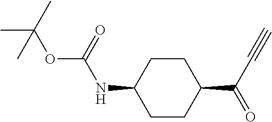

At 0° C., 49 ml of a 0.5 M solution of ethynyl magnesium bromide are added to a solution of 2.33 g of the compound obtained in the preceding stage, in 10 ml of THF. The medium is stirred at 0° C. for 4 h and then diluted in ethyl ether. The medium is run into ice-cold water and then separated by settling out. The aqueous phase is extracted twice in ether. The combined organic phases are dried over MgSO₄ and then evaporated so as to give 1.86 g of the expected compound.

### 5.3. Ethyl 6-((1s,4s)-4-(tert-butoxycarbonylamino)cyclohexyl)-2-(phenylamino)nicotinate

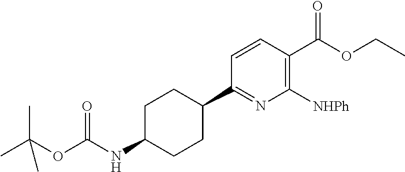

1.51 g of ethyl 3-amino-3-(phenylamino)acrylate are added to a solution of 1.85 g of the compound obtained in the preceding stage, in 150 ml of ethanol. The mixture is refluxed for 24 h. After a return to AT, the medium is concentrated so as to give 3.07 g of a brown oil.

### 5.4. 6-((1s,4s)-4-(tert-Butoxycarbonylamino)cyclohexyl)-2-(phenylamino)nicotinic acid

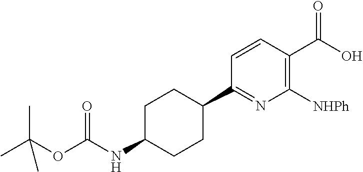

4.9 ml of 5M NaOH are added to a solution of 3.07 g of the compound obtained in the preceding stage, in 23 ml of ethanol. The medium is stirred at AT for 2 h and then concentrated and taken up in a mixture of water and ethyl ether. After separation by settling out, the aqueous phase is acidified to pH=5 with 6M HCl, and then extracted in DCM. The combined organic phases are dried over MgSO₄ and then evaporated so as to give 2.11 g of the expected compound.

### 5.5. tert-Butyl (1s,4s)-4-(6-(phenylamino)-5-(2-(piperidin-1-yl)ethylcarbamoyl)pyridin-2-yl)cyclohexylcarbamate

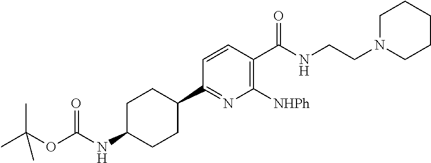

2.11 g of the compound obtained in the preceding stage are mixed with 0.66 g of 1-(2-aminoethyl)piperidine, 1.4 ml of triethylamine and 2.26 g of BOP in 51 ml of THF. The medium is stirred at AT for 24 h. The medium is hydrolyzed, diluted in DCM, washed with water, with a 10% Na₂CO₃ solution and with brine, then dried over MgSO₄ and then evaporated so as to give 3.14 g of a gum.

### 5.6. 6-((1s,4s)-4-Aminocyclohexyl)-2-(phenylamino)-N-(2-(piperidin-1-yl)ethyl)nicotinamide

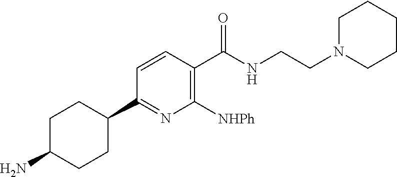

12.5 ml of a 4M solution of HCl in dioxane are added to a solution of 2.68 g of the compound obtained in the preceding stage, in 9 ml of dichloromethane. After stirring for 4 hours at AT, the medium is concentrated and then taken up in DCM. The organic phase is washed with a saturated solution of Na₂CO₃ and then with brine, then dried over MgSO₄ and then evaporated. 2.35 g of a mixture of the expected compound and of residual HMPA (by-product of BOP) originating from the preceding stage are obtained.

### 5.7. Bis-tert-butyl (1s,4s)-4-(6-(phenylamino)-5-(2-(piperidin-1-yl)ethylcarbamoyl)pyridin-2-yl)cyclohexylcarbamate

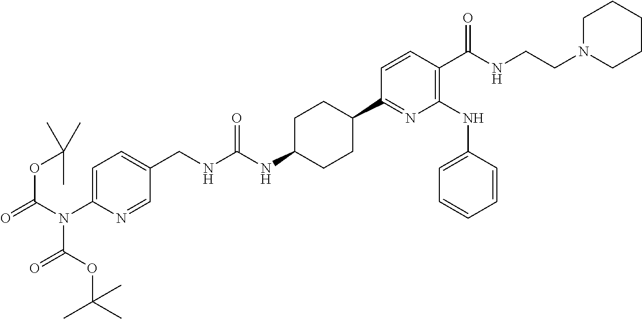

A solution of 1.19 g of the compound obtained in the preceding stage, in 40 ml of THF, is added, dropwise, to a solution of 430 mg of DMAP, and 0.86 g of DSC in 10 ml of THF. After stirring at AT for 15 min, 1.27 g of bis-tert-butyl 5-(aminomethyl)pyridin-2-ylcarbamate and 0.94 ml of triethylamine are added. After 3 h at AT, the medium is concentrated and then taken up in DCM. The organic phase is washed with a saturated solution of NaHCO₃ and then with brine, then dried over MgSO₄ and then evaporated. 2.09 g of a crude product are obtained, which product is purified by chromatography on silica gel (gradient: DCM to 9/1 DCM/MeOH) so as to give 0.18 g of the expected compound.

### 5.8. 6-((1s,4s)-4-(3-((6-Aminopyridin-3-yl)methyl)ureido)cyclohexyl)-2-(phenylamino)-N-(2-(piperidin-1-yl)ethyl)nicotinamide

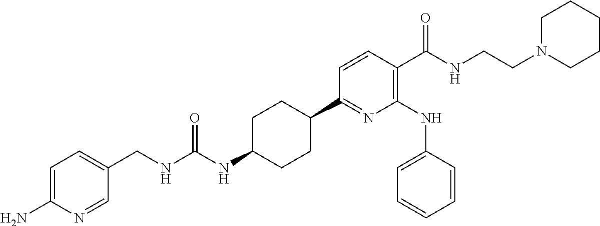

180 mg of the compound obtained in the preceding stage are solubilized in 0.35 ml of DCM and 0.35 ml of TFA is added. The medium is stirred at AT for 12 h and then concentrated. The crude product is triturated from a 10% solution of NaHCO₃, and the gum obtained is taken up in a 1/1 CHCl₃/MeOH mixture and the resulting product is evaporated. This crude product is purified by silica chromatography (gradient: DCM to DCM/MeOH/NH₄OH 80/20/1) to give and then taken up in acetone, filtered and evaporated so as to give 40 mg of a white powder: Mp=112° C.; ¹H NMR (250 MHz, DMSO-d6) 1.33-2.02 (m, 14H), 2.60-2.81 (m, 1 H), 3.17-3.42 (m, 6H), 3.64 (q, 2H), 3.72-3.86 (m, 1H), 4.05 (d, 2H), 6.13 (d, 1H), 6.20 (t, 1H), 6.67 (d, 1H), 6.72-6.89 (m, 3H), 6.95 (t, 1H), 7.30 (t, 2H), 7.56 (dd, 1H), 7.74 (d, 2H), 7.80 (d, 1H), 8.07 (d, 1H), 8.90 (t, 1H), 10.93 (s, 1H).

### Example 6

### 2-Ethylamino-N-methyl-6-{4-[3-(1-oxypyridin-3-ylmethyl)ureido]butyl}-nicotinamide (Compound No. 39).

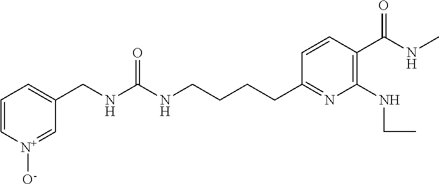

This compound is prepared according to the procedure described in Example 1.6. starting from the compound of preparation 2 and the compound of Example 1.5.

### ¹H NMR of the Compounds of Table 1

The δ chemical shifts are given in ppm.

| | |
|---|---|
| Compound | (250 MHz) 1.08 (t, 3 H), 1.25-1.44 (m, 2 H), 1.47-1.66 (m, 2 H), 2.47-2.55 (m, 2 H), 2.67 (d, 3 H), 2.97 |
| No. 1 | (q, 2 H), 3.28-3.45 (m, 2 H), 4.16 (d, 2 H), 5.92 (t, 1 H), 6.24-6.39 (m, 2 H), 7.27 (dd, 1 H), 7.58 (d, 1 |
| | H), 7.72 (d, 1 H), 8.16-8.35 (m, 2 H), 8.35-8.46 (m, 2 H) |
| 2 | (250 MHz) 0.25-0.41 (m, 2 H), 0.57-0.71 (m, 2 H), 1.26-1.46 (m, 2 H), 1.60 (quin, 2 H), 2.53 (t, 2 H), |
| | 2.66 (d, 3 H), 2.73-2.86 (m, 1 H), 2.88-3.06 (m, 2 H), 4.16 (d, 2 H), 5.93 (t, 1 H), 6.30 (t, 1 H), 6.41 (d, |
| | 1 H), 7.28 (dd, 1 H), 7.59 (dt, 1 H), 7.73 (d, 1 H), 8.19-8.33 (m, 1H), 8.33-8.48 (m, 3 H) |
| 3 | (250 MHz) 1.38 (qd, 2 H), 1.51-1.82 (m, 2 H), 2.63 (t, 2 H), 2.75 (d, 3 H), 3.00 (q, 2 H), 4.16 (d, 2 H), |
| | 5.96 (t, 1 H), 6.31 (t, 1 H), 6.66 (d, 1 H), 6.89 (t, 1 H), 7.17-7.36 (m, 3 H), 7.61 (d, 1 H), 7.69 (d, 2 H), |
| | 7.95 (d, 1 H), 8.42 (d, 2 H), 8.60 (d, 1 H), 11.04 (s, 1 H) |
| 4 | (250 MHz) 1.14 (t, 3 H), 1.29-1.43 (m, 4 H), 1.43-1.54 (m, 4 H), 1.54-1.72 (m, 2 H), 2.28-2.47 (m, 6 |
| | H), 2.52-2.62 (m, 2 H), 2.96-3.09 (m, 2 H), 3.24-3.34 (m, 2 H), 3.33-3.49 (m, 2 H), 3.97 (d, 2 H), |
| | 5.75 (s, 2 H), 5.82 (t, 1 H), 6.04 (t, 1 H), 6.39 (dd, 2 H), 7.26 (dd, 1 H), 7.68-7.87 (m, 2 H), 8.18-8.37 |
| | (m, 2 H) |
| 5 | (250 MHz) 1.14 (t, 3 H), 1.27-1.49 (m, 2 H), 1.52-1.75 (m, 2 H), 2.51-2.60 (m, 2 H), 2.72 (d, 3 H), |
| | 3.01 (q, 2 H), 3.34-3.48 (m, 2 H), 3.97 (d, 2 H), 5.75 (s, 2 H), 5.83 (t, 1 H), 6.05 (t, 1 H), 6.39 (d, 2 H), |
| | 7.26 (dd, 1 H), 7.70-7.85 (m, 2 H), 8.20-8.41 (m, 2 H) |
| 6 | (250 MHz) 1.14 (t, 3 H), 1.29-1.48 (m, 2 H), 1.49-1.75 (m, 2 H), 2.00 (s, 3H), 2.50-2.61 (m, 2 H), |
| | 2.72 (d, 3 H), 3.01 (q, 2 H), 3.32-3.47 (m, 2 H), 3.97 (d, 2 H), 5.54 (s, 2 H), 5.80 (t, 1 H), 6.03 (t, 1 H), |
| | 6.39 (d, 1 H), 7.13 (s, 1 H), 7.67 (s, 1 H), 7.77 (d, 1 H), 8.21-8.44 (m, 2 H) |
| 7 | (250 MHz) 1.42 (m, 2 H), 1.70 (quin, 2 H), 2.67 (t, 2 H), 2.80 (d, 3 H), 3.04(q, 2 H), 3.98 (d, 2 H), 5.69- |
| | 5.94 (m, 3 H), 6.06 (t, 1 H), 6.40 (d, 1 H), 6.71 (d, 1 H), 6.94 (t, 1 H), 7.22-7.39 (m, 3 H), 7.67-7.89 (m, |
| | 3 H), 8.00 (d, 1 H), 8.64 (d, 1 H), 11.08 (s, 1 H) |
| 8 | (250 MHz) 1.08 (t, 3 H), 2.50 (t, 2 H), 2.68 (d, 3 H), 3.19 (q, 2 H), 3.28-3.39 (m, 2 H), 4.19 (d, 2 H), 6.19 |
| | (t, 1 H), 6.48-6.66 (m, 2 H), 7.29 (br. s., 1 H), 7.61 (d, 1 H), 7.77 (d, 1H), 8.25-8.74 (m, 4 H) |
| 9 | (250 MHz) 0.27-0.41 (m, 2 H), 0.58-0.73 (m, 2 H), 2.52 (t, 2 H), 2.66 (d, 3 H), 3.10-3.25 (m, 3 H), |
| | 4.20 (d, 2 H), 6.21 (t, 1 H), 6.55 (t, 1 H), 6.66 (d, 1 H), 7.17-7.72 (m, 3 H), 7.78 (d, 1 H), 8.32-8.59 (m, |
| | 3 H) |
| 10 | (400 MHz) 2.60 (t, 2 H), 2.81 (d, 3 H), 3.23-3.31 (m, 2 H), 4.24 (d, 2 H), 6.26 (t, 1 H), 6.60 (t, 1 H), 6.94 |
| | (d, 1 H), 6.98 (t, 1 H), 7.27-7.38 (m, 3 H), 7.59-7.71 (m, 3 H), 8.03 (d, 1 H), 8.46 (br. s., 2 H), 8.66- |
| | 8.83 (m, 1 H), 11.03 (s, 1 H) |
| 11 | (250 MHz) 1.16 (d, 6 H), 1.28-1.49 (m, 2 H), 1.51-1.74 (m, 2 H), 2.51-2.63 (m, 2 H), 2.72 (d, 3 H), |
| | 3.01 (q, 2 H), 3.97 (d, 2 H), 4.09-4.34 (m, 1 H), 5.75 (s, 2 H), 5.81 (t, 1 H), 6.04 (t, 1 H), 6.38 (dd, 2 H), |
| | 7.26 (dd, 1 H), 7.72-7.87 (m, 2 H), 8.22-8.43 (m, 2 H) |
| 12 | (250 MHz) 1.16 (d, 6 H), 1.28-1.48 (m, 2 H), 1.52-1.73 (m, 2 H), 2.51-2.59 (m, 2 H), 2.71 (d, 3 H), |
| | 2.91-3.14 (m, 8 H), 4.02 (d, 2 H), 4.09-4.31 (m, 1 H), 5.83 (t, 1 H), 6.08 (t, 1 H), 6.38 (d, 1 H), 6.58 (d, |
| | 1 H), 7.40 (d, 1 H), 7.77 (d, 1 H), 7.96 (s, 1 H), 8.16-8.47 (m, 2 H) |
| 13 | (400 MHz) 1.16 (d, 6 H), 1.32-1.46 (m, 2 H), 1.53-1.67 (m, 2 H), 2.42 (s, 3 H), 2.54 (t, 2 H), 2.72 (d, 3 |
| | H), 2.96-3.07 (m, 2 H), 4.09-4.29 (m, 3 H), 5.94 (t, 1 H), 6.28 (t, 1 H), 6.37 (d, 1 H), 7.16 (d, 1 H), 7.50 |
| | (dd, 1 H), 7.77 (d, 1 H), 8.23-8.41 (m, 3 H) |
| 14 | (400 MHz) 1.16 (d, 6 H), 1.32-1.52 (m, 2 H), 1.55-1.74 (m, 2 H), 2.26 (s, 3 H), 2.55 (t, 2 H), 2.72 (d, 3 |
| | H), 2.95-3.13 (m, 2 H), 4.10-4.30 (m, 3 H), 5.96 (t, 1 H), 6.31 (t, 1 H), 6.37 (d, 1 H), 7.43 (s, 1 H), 7.76 |
| | (d, 1 H), 8.18-8.39 (m, 4 H) |
| 15 | (250 MHz) 1.31-1.44(m, 2 H), 1.44-1.73 (m, 8 H), 1.73-1.97 (m, 4 H), 2.37-2.53 (m, 6 H), 2.65- |
| | 2.82 (m, 1 H), 3.40 (q, 2 H), 3.70-3.86 (m, 1 H), 3.99 (d, 2 H), 5.76 (s, 2 H), 5.92-6.05 (m, 2 H), 6.39 |
| | (d, 1 H), 6.90 (d, 1 H), 7.18-7.30 (m, 2 H), 7.50 (t, 1 H), 7.75 (d, 1 H), 7.79 (d, 1 H), 8.10 (d, 1 H), 8.49 |
| | (s, 1 H), 8.69 (t, 1 H), 11.26 (s, 1 H) |
| 16 | (250 MHz) 1.51-1.95 (m, 8 H), 2.61-2.89 (m, 3 H), 2.86-3.18 (m, 8 H), 3.37-3.55 (m, 2 H), 3.70- |
| | 3.89 (m, 1 H), 4.01 (d, 2 H), 5.93-6.10 (m, 2 H), 6.23 (br. s., 2 H), 6.44-6.59 (m, 1 H), 6.91 (d, 1 H), |
| | 7.27 (d, 1 H), 7.33-7.45 (m, 1 H), 7.50 (t, 1 H), 7.74 (d, 1 H), 7.79 (s, 1 H), 8.10 (d, 1 H), 8.52 (s, 1 H), |
| | 8.70 (t, 1 H), 11.26 (s, 1 H) |
| 17 | (250 MHz) 1.33-2.02 (m, 14 H), 2.60-2.81 (m, 1 H), 3.17-3.42 (m, 6 H), 3.64 (q, 2 H), 3.72-3.86 |
| | (m, 1 H), 4.05 (d, 2 H), 6.13 (d, 1 H), 6.20 (t, 1 H), 6.67 (d, 1 H), 6.72-6.89 (m, 3 H), 6.95 (t, 1 H), 7.30 |
| | (t, 2 H), 7.56 (dd, 1 H), 7.74 (d, 2 H), 7.80 (d, 1 H), 8.07 (d, 1 H), 8.90 (t, 1H), 10.93 (s, 1 H) |
| 18 | (400 MHz) 1.15 (t, 3 H), 1.32-1.45 (m, 2 H), 1.57-1.69 (m, 2 H), 2.55 (t, 2H), 3.01 (q, 2 H), 3.26 (s, 3 |
| | H), 3.33-3.51 (m, 6 H), 3.98 (d, 2 H), 5.75 (s, 2 H), 5.83 (t, 1 H), 6.05 (t, 1H), 6.33-6.46 (m, 2 H), 7.25 |
| | (dd, 1 H), 7.77 (s, 1 H), 7.81 (d, 1 H), 8.32 (t, 1 H), 8.37 (t, 1 H) |
| 19 | (250 MHz) 1.14 (t, 3 H), 1.30-1.49 (m, 2 H), 1.52-1.78 (m, 2 H), 2.56 (t, 2H), 2.64 (t, 2 H), 2.88-3.17 |
| | (m, 10 H), 3.22-3.50 (m, 4 H), 3.98 (d, 2 H), 5.76 (s, 2 H), 5.82 (t, 1 H), 6.05 (t, 1 H), 6.34-6.46 (m, 2 |
| | H), 7.27 (dd, 1 H), 7.73-7.89 (m, 2 H), 8.19-8.37 (m, 2 H) |
| 20 | (250 MHz) 1.28-1.59 (m, 8 H), 1.71 (quin, 2 H), 2.31-2.48 (m, 6 H), 2.67 (t, 2 H), 3.04 (q, 2 H), 3.38 (q, |
| | 2 H), 3.98 (d, 2 H), 5.74 (s, 2 H), 5.84 (t, 1 H), 6.05 (t, 1 H), 6.38 (d, 1H), 6.72 (d, 1 H), 6.89-7.04 (m, 1 |
| | H), 7.21-7.38 (m, 3 H), 7.73 (d, 2 H), 7.79 (d, 1 H), 8.00 (d, 1 H), 8.57 (t, 1 H), 10.99 (s, 1 H) |
| 24 | (400 MHz) 1.15 (t, 3 H), 1.33-1.43 (m, 2 H), 1.56-1.68 (m, 2 H), 2.54 (d, 4H), 2.64-2.75 (m, 4 H), 2.81-2.97 |
| | (m, 4 H), 3.01 (q, 2 H), 3.27-3.36 (m, 2 H), 3.39 (dd, 2 H), 3.97 (d, 2 H), 5.75 (s, 2 H), 5.82 (t, 1 H), |
| | 6.05 (t, 1 H), 6.39 (t, 2 H), 7.26 (dd, 1 H), 7.74-7.80 (m, 2 H), 8.23-8.34 (m, 2 H) |
| 27 | (250 MHz) 1.14 (t, 3 H), 1.20-1.46 (m, 6 H), 1.56-1.74 (m, 2 H), 2.52-2.58 (m, 2 H), 2.72 (d, 3 H), 2.96 |
| | (q, 2 H), 3.33-3.46 (m, 2 H), 3.98 (d, 2 H), 5.74 (s, 2 H), 5.81 (t, 1 H), 6.06 (t, 1 H), 6.39 (d, 2 H), 7.26 |
| | (dd, 1 H), 7.73-7.86 (m, 2 H), 8.22-8.48 (m, 2 H) |
| 28 | (250 MHz) 1.14 (t, 3 H), 1.38 (tdd, 2 H), 1.62 (dq, 2 H), 2.52-2.64 (m, 2 H), 3.01 (q, 2 H), 3.21-3.59 (m, |
| | 6 H), 3.98 (d, 2 H), 4.69 (t, 1 H), 5.74 (s, 2 H), 5.82 (t, 1 H), 6.05 (t, 1 H), 6.34-6.46 (m, 2 H), 7.26 (dd, 1 |
| | H), 7.78 (d, 1 H), 7.83 (d, 1 H), 8.23-8.40 (m, 2 H) |
| 29 | (400 MHz) 0.53 (s, 2 H), 0.67 (d, 2 H), 1.15 (t, 3H), 1.31-1.45(m, 2 H), 1.53-1.71 (m, 2 H), 2.50-2.61 |
| | (m, 2 H), 2.70-2.86 (m, 1 H), 3.00 (q, 2 H), 3.35-3.46 (m, 2 H), 3.97(d, 2 H), 5.67-5.78 (m, 2 H), 5.82 |
| | (br. s., 1 H), 6.05 (br. s., 1 H), 6.32-6.48 (m, 2 H), 7.25 (d, 1 H), 7.69-7.86 (m, 2 H), 8.24-8.44 (m, 2 H) |
| 31 | (400 MHz) 0.90 (t, 3 H), 1.14 (t, 3 H), 1.26-1.53 (m, 6 H), 1.62 (quin, 2 H), 2.54 (t, 2 H), 3.01 (q, 2 H), |
| | 3.20 (q, 2 H), 3.34-3.45 (m, 2 H), 3.98 (d, 2 H), 5.84 (t, 1 H), 5.93 (br. s., 2H), 6.07 (t, 1 H), 6.33-6.47 |
| | (m, 2 H), 7.30 (dd, 1 H), 7.72-7.85 (m, 2 H), 8.22-8.39 (m, 2 H) |
| 33 | (250 MHz) 1.13 (t, 3 H), 1.40 (quin, 2 H), 1.62 (quin, 2 H), 2.50-2.61 (m, 2H), 2.72 (d, 3 H), 3.02 (q, 2 |
| | H), 3.33-3.50 (m, 2 H), 4.25 (d, 2 H), 6.05 (t, 1 H), 6.33-6.53 (m, 2 H), 7.53 (d, 1 H), 7.77 (d, 1 H), 8.21-8.58 |
| | (m, 4H) |
| 34 | (400 MHz) 1.31 (s, 6 H), 1.75-1.86 (m, 2 H), 2.77-2.88 (m, 5 H), 3.94 (d, 2H), 5.68 (t, 1 H), 5.74 (s, 2 |
| | H), 6.01 (t, 1 H), 6.36 (d, 1 H), 6.87 (d, 1 H), 6.94 (t, 1 H), 7.23 (dd, 1 H), 7.30 (t, 2 H), 7.72 (d, 2 H), 7.75 |
| | (d, 1 H), 8.03 (d, 1 H), 8.66 (q, 1 H), 11.05 (s, 1 H) |
| 35 | (400 MHz) 1.14 (t, 3 H), 1.34-1.53 (m, 2 H), 1.65 (quin, 2 H), 2.56 (t, 2 H), 2.72 (d, 3 H), 3.07 (q, 2 H), |
| | 3.35-3.47 (m, 2 H), 5.48 (s, 2 H), 6.00 (d, 1 H), 6.31-6.46 (m, 2 H), 7.31-7.44 (m, 1 H), 7.76 (d, 1 H), |
| | 7.86 (d, 2 H), 8.23-8.45 (m, 2 H) |
| 36 | (400 MHz) 1.14 (t, 3 H), 1.37 (quin, 2 H), 1.62 (quin, 2 H), 2.45 (t, 2 H), 2.54 (t, 2 H), 2.72 (d, 3 H), 2.99 |
| | (q, 2 H), 3.11 (q, 2 H), 3.35-3.45 (m, 2 H), 5.66 (s, 2 H), 5.71 (t, 1 H), 5.84(t, 1 H), 6.34-6.43 (m, 2 H), |
| | 7.20 (dd, 1 H), 7.71 (d, 1 H), 7.76 (d, 1 H), 8.24-8.38 (m, 2 H) |
| 37 | (250 MHz) 0.33-0.45 (m, 2 H), 0.63-0.74 (m, 2 H), 2.72 (d, 3 H), 2.74-2.83 (m, 1 H), 3.01-3.09 (m, 2 |
| | H), 3.11-3.20 (m, 2 H), 3.99 (d, 2 H), 5.75-6.00 (m, 2 H), 6.24 (t, 1H), 6.25-6.48 (m, 3 H), 6.51-6.61 |
| | (m, 2 H), 7.41 (d, 1 H), 7.77 (s, 1 H), 7.85 (d, 1 H), 8.33-8.44 (m, 1 H), 8.48 (s, 1 H) |
| 39 | (250 MHz) 1.14 (t, 3 H), 1.32-1.47 (m, 2 H), 1.55-1.70 (m, 2 H), 2.53 (t, 2H), 2.72 (d, 3 H), 3.03 (q, 2 |
| | H), 3.33-3.46 (m, 2 H), 4.15 (d, 2 H), 6.08 (t, 1 H), 6.34-6.47 (m, 2 H), 7.20 (d, 1 H), 7.30-7.40 (m, 1 |
| | H), 7.77 (d, 1 H), 8.03-8.12 (m, 2 H), 8.24-8.40 (m, 2 H) |
| 40 | (400 MHz) 1.14 (t, 3 H), 1.40 (quin, 2 H), 1.63 (quin, 2 H), 2.55 (t, 2 H), 2.72(d, 3 H), 3.02 (q, 2 H), 3.39 |
| | (quin, 2 H), 4.30 (d, 2 H), 6.12 (t, 1 H), 6.38 (d, 1 H), 6.48 (t, 1 H), 7.76 (d, 1 H), 7.85 (d, 1 H), 7.97 (d, 1 |
| | H), 8.24-8.40 (m, 2 H), 8.62 (s, 1 H) |

TABLE I

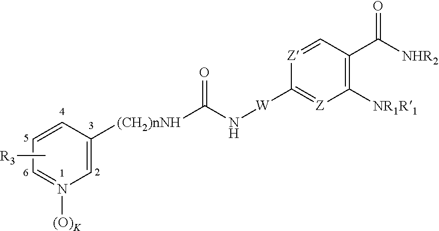

| Compound No. | R₃ | n | k | Z/Z′ | W | R₁ | R′₁ | R₂ | MS (MH⁺) | LC (method) | Mp (° C.)/NMR | Scheme |
|---|---|---|---|---|---|---|---|---|---|---|---|---|
| 1 | H | 1 | 0 | N/CH | —(CH₂)₄— | Et | H | —Me | 385 | 4.1 (TFA15) | NMR | 5 |
| 2 | H | 1 | 0 | N/CH | —(CH₂)₄— | Cy | H | —Me | 397 | 4.09 (TFA15) | NMR | 5 |
| 3 | H | 1 | 0 | N/CH | —(CH₂)₄— | 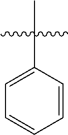 | H | —Me | 433 | 5.82 (TFA15) | NMR | 5; see ex. 3.6 |
| 4 | 6-NH₂ | 1 | 0 | N/CH | —(CH₂)₄— | Et | H | 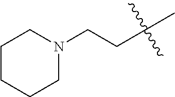 | 497 | 0.54 (TFA3) | NMR | 4 |
| 5 | 6-NH₂ | 1 | 0 | N/CH | —(CH₂)₄— | Et | H | —Me | 400 | 0.53 (TFA3) | NMR | 4; see ex.1 |
| 6 | 5-Me, 6-NH₂ | 1 | 0 | N/CH | —(CH₂)₄— | Et | H | —Me | 414 | 0.8 (TFA3) | NMR | 4 |
| 7 | 6-NH₂ | 1 | 0 | N/CH | —(CH₂)₄— | 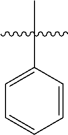 | H | —Me | 448 | 0.78 (TFA3) | NMR | 4 |
| 8 | H | 1 | 0 | N/CH | —(CH₂)₂C≡C— | Et | H | —Me | 381 | 4.71 (TFA15) | NMR | 3 |
| 9 | H | 1 | 0 | N/CH | —(CH₂)₂C≡C— | Cy | H | —Me | 393 | 4.74 (TFA15) | NMR | 3 |
| 10 | H | 1 | 0 | N/CH | —(CH₂)₂C≡C— | 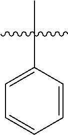 | H | —Me | 429 | 4.96 (TFA15) | NMR | 3; see ex. 3.5 |
| 11 | 6-NH₂ | 1 | 0 | N/CH | —(CH₂)₄— | i-Pr | H | —Me | 414 | 0.60 (TF3) | NMR | 4 |
| 12 | 6-NMe₂ | 1 | 0 | N/CH | —(CH₂)₄— | i-Pr | H | —Me | 442 | 0.64 (TFA3) | NMR | 4 |
| 13 | 6-Me | 1 | 0 | N/CH | —(CH₂)₄— | i-Pr | H | —Me | 413 | 0.60 (TFA3) | NMR | 4 |
| 14 | 5-Me | 1 | 0 | N/CH | —(CH₂)₄— | i-Pr | H | —Me | 413 | 0.61 (TFA3) | NMR | 4 |
| 15 | 6-NH₂ | 1 | 0 | N/CH | 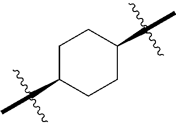 | 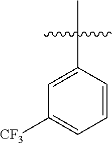 | H | 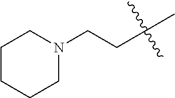 | 639 | 1.07 (TFA3) | NMR | 8 |
| 16 | 6-NH₂ | 1 | 0 | N/CH | 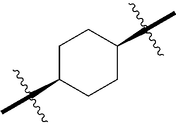 | 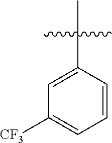 | H | 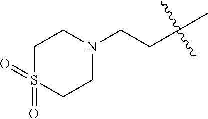 | 689 | 1.07 (TFA3) | NMR | 8 |
| 17 | 6-NH₂ | 1 | 0 | N/CH | 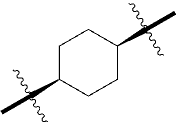 | 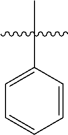 | H | 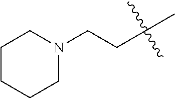 | 571 | 0.88 (TFA3) | NMR | 8 |
| 18 | 6-NH₂ | 1 | 0 | N/CH | —(CH₂)₄— | Et | H | —CH₂CH₂OMe | 444 | 6.16 (TFA15) | NMR | 5 |
| 19 | 6-NH₂ | 1 | 0 | N/CH | —(CH₂)₄— | Et | H | 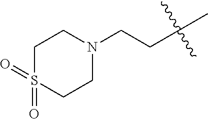 | 547 | 0.53 (TFA3) | NMR | 5; see ex.2 |
| 20 | 6-NH₂ | 1 | 0 | N/CH | —(CH₂)₄— | 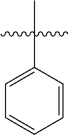 | H | 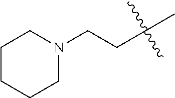 | 545 | 1.23 (TFA3) | NMR | 4 |
| 21 | 6-NH₂ | 1 | 0 | N/CH | —(CH₂)₄— | Cy | H | —Me | | | | |
| 22 | 6-NH₂ | 1 | 0 | N/CH | —(CH₂)₄— | i-Pr | H | 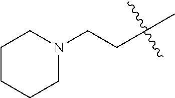 | | | | |
| 23 | 6-NH₂ | 1 | 0 | N/CH | —(CH₂)₄— | Cy | H | 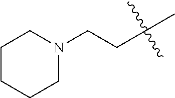 | | | | |
| 24 | 6-NH₂ | 1 | 0 | N/CH | —(CH₂)₄— | Et | H | 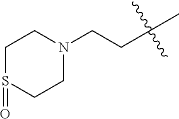 | 531 | 0.44 (TFA3) | NMR | 5 |
| 25 | 6-NH₂ | 1 | 0 | N/N | —(CH₂)₄— | Et | H | —Me | | | | |
| 26 | 6-NH₂ | 1 | 0 | CH/CH | —(CH₂)₄— | Et | H | —Me | | | | |
| 27 | 6-NH₂ | 1 | 0 | N/CH | —(CH₂)₆— | Et | H | —Me | 428 | 0.63 (TFA3) | NMR | 5 |
| 28 | 6-NH₂ | 1 | 0 | N/CH | —(CH₂)₄— | Et | H | —CH₂CH₂OH | 430 | 0.49 (TFA3) | NMR | 5 |
| 29 | 6-NH₂ | 1 | 0 | N/CH | —(CH₂)₄— | Et | H | —Cy | | | | |
| 30 | 6-NH₂ | 1 | 0 | N/CH | —(CH₂)₄— | Et | H | 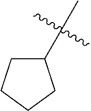 | 454 | 0.82 (TFA3) | NMR | 5 |
| 31 | 6-NH₂ | 1 | 0 | N/CH | —(CH₂)₄— | Et | H | -(n-Bu) | 442 | 0.81 (TFA3) | NMR | 5 |
| 32 | 5-NH₂ | 1 | 0 | N/CH | —(CH₂)₄— | Et | H | —Me | | | | |
| 33 | 5-F | 1 | 0 | N/CH | —(CH₂)₄— | Et | H | —Me | 403 | 0.64 (TFA3) | NMR | 4 |
| 34 | 6-NH₂ | 1 | 0 | N/CH | —(CH₂)₂CMe₂— | 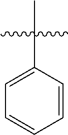 | H | —Me | 462 | 1.12 (TFA3) | NMR | 9 |
| 35 | 6-NH₂ | 0 | 0 | N/CH | —(CH₂)₄— | Et | H | —Me | 386 | 0.56 (TFA3) | NMR | 4 |
| 36 | 6-NH₂ | 2 | 0 | N/CH | —(CH₂)₄— | Et | H | —Me | 414 | 0.56 (TFA3) | NMR | 4 |
| 37 | 6-NH₂ | 1 | 0 | N/CH | —CH₂CH≡CH— | Cy | H | —Me | 410 | 0.54 (TFA3) | NMR | 6 |
| | | | | | form Z | | | | | | | |
| 38 | 6-NH₂ | 1 | 0 | N/CH | 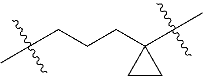 | 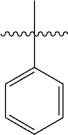 | H | —Me | | | | 9 |
| 39 | H | 1 | 1 | N/CH | —(CH₂)₄— | Et | H | —Me | 401 | 0.55 (TFA3) | NMR | 4 |
| 40 | 6-CN | 1 | 0 | N/CH | —(CH₂)₄— | Et | H | —Me | 410 | 0.71 (TFA3) | NMR | 4 |

n-Bu: n-butyl; t-Bu: tert-butyl; i-Pr: isopropyl; Cy: cyclopropyl

TABLE II
Chemical name of some compounds of Table I (obtained from the Autonom ® software):

| | |
|---|---|
| 1 | 2-Ethylamino-N-methyl-6-[4-(3-pyridin-3-ylmethylureido)butyl]nicotinamide |
| 2 | 2-Cyclopropylamino-N-methyl-6-[4-(3-pyridin-3-ylmethylureido)butyl]nicotinamide |
| 3 | N-Methyl-2-phenylamino-6-[4-(3-pyridin-3-ylmethylureido)butyl]nicotinamide |
| 4 | 6-{4-[3-(6-Aminopyridin-3-ylmethyl)ureido]butyl}-2-ethylamino-N-(2-piperidin-1-yl-ethyl)nicotinamide |
| 5 | 6-{4-[3-(6-Aminopyridin-3-ylmethyl)ureido]butyl}-2-ethylamino-N-methylnicotinamide |
| 6 | 6-{4-[3-(6-Amino-5-methylpyridin-3-ylmethyl)ureido]butyl}-2-ethylamino-N-methylnicotinamide |
| 7 | 6-{4-[3-(6-Aminopyridin-3-ylmethyl)ureido]butyl}-N-methyl-2-phenylaminonicotinamide |
| 8 | 2-Ethylamino-N-methyl-6-[4-(3-pyridin-3-ylmethylureido)but-1-ynyl]nicotinamide |
| 9 | 2-Cyclopropylamino-N-methyl-6-[4-(3-pyridin-3-ylmethylureido)but-1-ynyl]nicotinamide |
| 10 | N-Methyl-2-phenylamino-6-[4-(3-pyridin-3-ylmethylureido)but-1-ynyl]nicotinamide |
| 11 | 6-{4-[3-(6-Aminopyridin-3-ylmethyl)ureido]butyl}-2-isopropylamino-N-methylnicotinamide |
| 12 | 6-{4-[3-{6-Dimethylaminopyridin-3-ylmethyl)ureido]butyl}-2-isopropylamino-N-methylnicotinamide |
| 13 | 2-Isopropylamino-N-methyl-6-{4-[3-(6-methylpyridin-3-ylmethyl)ureido]butyl}nicotinamide |
| 14 | 2-Isopropylamino-N-methyl-6-{4-[3-(5-methylpyridin-3-ylmethyl)ureido]butyl}nicotinamide |
| 15 | 6-{cis-4-[3-(6-Aminopyridin-3-ylmethyl)ureido]cyclohexyl}-N-(2-piperidin-1-ylethyl)-2-(3-trifluoromethylphenylamino)- |
| | nicotinamide |
| 16 | 6-[cis-4-({[(6-Aminopyridin-3-yl)methyl]carbamoyl}amino)cyclohexyl]-N-[2-(1,1-dioxidothiomorpholin-4-yl)ethyl]-2-{[3- |
| | (trifluoromethyl)phenyl]amino}nicotinamide |
| 17 | 6-{cis-4-[3-(6-Aminopyridin-3-ylmethyl)ureido]cyclohexyl}-2-phenylamino-N-(2-piperidin-1-ylethyl)nicotinamide |
| 18 | 6-{4-[3-(6-Aminopyridin-3-ylmethyl)ureido]butyl}-2-ethylamino-N-(2-methoxyethyl)nicotinamide |
| 19 | 6-[4-({[(6-Aminopyridin-3-yl)methyl]carbamoyl}amino)butyl]-N-[2-(1,1-dioxidothiomorpholin-4-yl)ethyl]-2- |
| | (ethylamino)nicotinamide |
| 20 | 6-{4-[3-(6-Aminopyridin-3-ylmethyl)ureido]butyl}-2-phenylamino-N-(2-piperidin-1-yl-ethyl)nicotinamide |
| 24 | 6-{4-[3-(6-Aminopyridin-3-ylmethyl)ureido]butyl}-2-ethylamino-N-[2-(1-oxo-1 lambda 4-thiomorpholin-4- |
| | yl)ethyl]nicotinamide |
| 27 | 6-{6-[3-(6-Aminopyridin-3-ylmethyl)ureido]hexyl}-2-ethylamino-N-methylnicotinamide |
| 28 | 6-{4-[3-(6-Aminopyridin-3-ylmethyl)ureido]butyl}-2-ethylamino-N-(2-hydroxyethyl)nicotinamide |
| 29 | 6-[4-[3-(6-Aminopyridin-3-ylmethyl)ureido]butyl}-N-cyclopropyl-2-ethylaminonicotinannide |
| 31 | 6-{4-[3-(6-Aminopyridin-3-ylmethyl)ureido]butyl}-N-butyl-2-ethylaminonicotinamide |
| 33 | 2-Ethylamino-6-{4-[3-(5-fluoropyridin-3-ylmethyl)ureido]butyl}-N-methylnicotinamide |
| 34 | 6-{3-[3-(6-Aminopyridin-3-ylmethyl)ureido]-1,1-dimethylpropyl}-N-methyl-2-phenylaminonicotinamide |
| 35 | 6-{4-[3-(6-Aminopyridin-3-yl)ureido]butyl}-2-ethylamino-N-methylnicotinamide |
| 36 | 6-(4-{3-[2-(6-Aminopyridin-3-yl)ethyl]ureido}butyl)-2-ethylamino-N-methylnicotinamide |
| 37 | 6-{(Z)-4-[3-(6-Aminopyridin-3-ylmethyl)ureido]but-1-enyl}-2-cyclopropylamino-N-methylnicotinainide |
| 39 | 2-Ethylamino-N-methyl-6-{4-[3-(1-oxypyridin-3-ylmethyl)ureido]butyl}nicotinamide |
| 40 | 6-{4-[3-(6-Cyanopyridin-3-ylmethyl)ureido]butyl}-2-ethylamino-N-methylnicotinamide |

The compounds described in Table II were subjected to pharmacological tests for determining the anticancer activity. They were tested in vitro on the following tumour lines: HCT116 (ATCC-CCL247) and PC3 (ATCC-CRL 1435). The proliferation and the cell viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology 2003, 64(4), 399-406. In this test, the mitochondrial capacity of the living cells for converting MTS into a coloured compound after incubation of the tested compound for 72 hours is measured. IC₅₀ denotes the concentration of compound which results in a 50% loss of proliferation and of cell viability.

For the compounds of Table I, an IC₅₀<1000 nM (1 μM) was found on the HCT116 and PC3 lines.

1. A compound of formula (I): 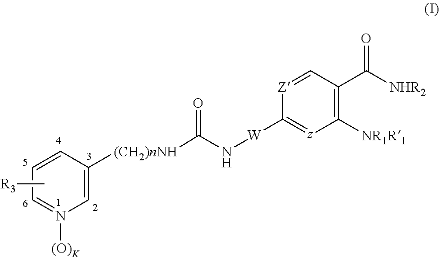
in which:
when Z and Z′ represent respectively N or CH, W represents a —(C₁-C₄)alkylene-CH₂CH₂—, —(C₁-C₄)alkylene-CH═CH— or —(C₁-C₄)alkylene-C≡C— group in which the (C₁-C₄)alkylene group is linked to the —C(═O)—NH— group;
when Z and Z′ represent respectively N and CH, W represents:
a 1,4-cyclohexylene group 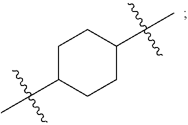
a —(CH₂)₁₋₄CRR′-group in which the (CH₂)₁₋₄ group is linked to the —C(═O)—NH— group and R and R′ represent a fluorine atom or a (C₁-C₄)alkyl group or form, together with the carbon atom to which they are attached, a cyclopropyl group;
k is an integer equal to 0 or 1;
n is an integer equal to 0, 1 or 2;
R₁ represents a hydrogen atom, a (C₁-C₆)alkyl group, a (C₃-C₆)cycloalkyl group or a phenyl group optionally substituted with a trifluoromethyl group;
R′₁ represents a hydrogen atom or a (C₁-C₆)alkyl group;
R₂ represents:
a (C₃-C₆)cycloalkyl group;
a (C₁-C₆)alkyl group optionally substituted with:
one or more hydroxyl or (C₁-C₄)alkoxy groups;
an NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-, and
being optionally substituted with one or more substituent(s), which may be identical to or different from one another when there are several thereof, chosen from an —OH; (C₁-C₄)alkoxy or (C₁-C₄)alkyl group;
R₃ represents at least one substituent of the pyridine nucleus, chosen from a hydrogen or fluorine atom, a (C₁-C₄)alkyl group, a cyano or —NR_{c}R_{d} in which R_{c} and R_{d} represent a hydrogen atom or a (C₁-C₄)alkyl group.

2. The compound according to claim 1, in which Z and Z′ represent respectively N and CH; CH and CH, or N and N when W represents a —(C₁-C₄)alkylene-CH₂CH₂—, —(C₁-C₄)alkylene-CH═CH— or —(C₁-C₄)alkylene-C≡C— group.

3. The compound according to claim 1 wherein W represents the 1,4-cis or trans-cyclohexylene group or else the —(C₁-C₄)alkylene CH═CH— group in E or Z form.

4. The compound according to claim 1 wherein R′₁ represents a hydrogen atom.

5. The compound according to claim 1 wherein R₂ represents:
a cyclopropyl or cyclopentyl group; or
a (C₁-C₆)alkyl group; or
a (C₁-C₆)alkyl group substituted with one or more hydroxyl or (C₁-C₄)alkoxy groups; or
a (C₁-C₆)alkyl group substituted with a (C₄-C₆)heterocycloalkyl group chosen from a

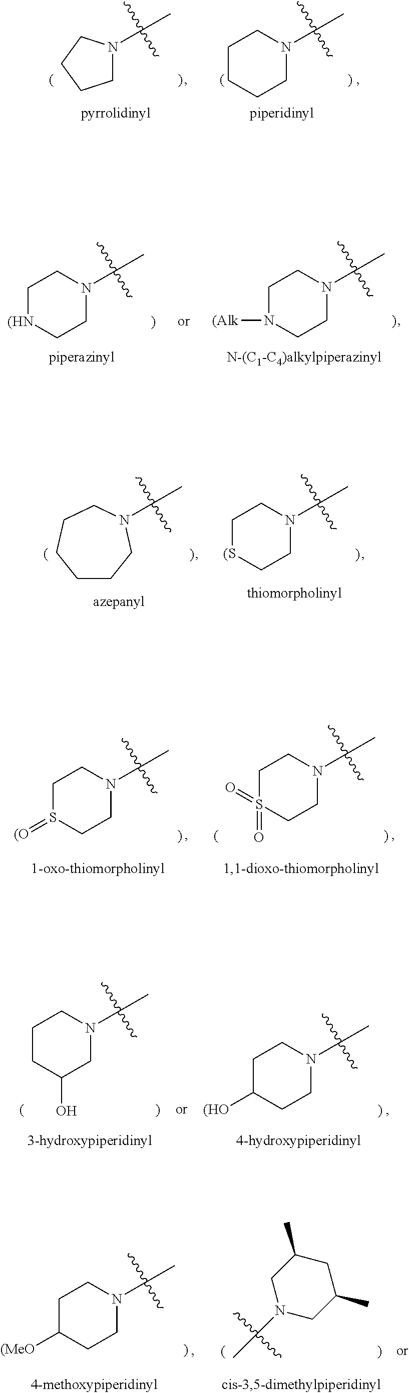

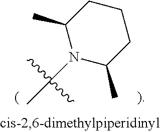

6. The compound according to claim 1, in which R₁ represents a cyclopropyl group or a phenyl group optionally substituted with a trifluoromethyl group.

7. The compound according to claim 1, in which the number of substituents R₃ is equal to 1 and/or R₃ is in position 5 or 6 on the pyridine nucleus.

8. The compound according to claim 1, in which R₃ is H, —NH₂ or CN.

9. The compound according to claim 1, of formula (I′): 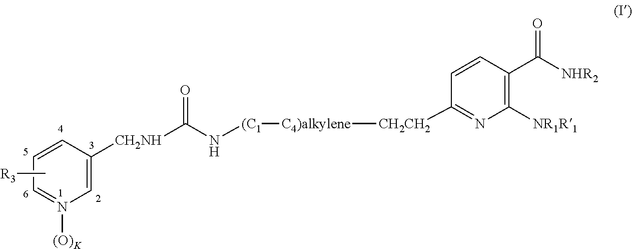
in which k, R₁, R′₁, R₂ and R₃ are as defined in claim 1.

10. The compound according to claim 1, of formula (I″): 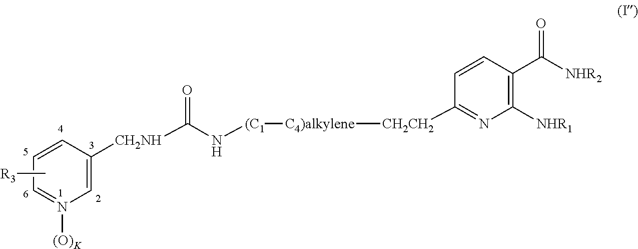
in which k is 0 or 1, R₁ represents a (C₁-C₄)alkyl group, R₂ represents a (C₁-C₆)alkyl group optionally substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, and R₃ is positioned in position 5 or 6.

11. The compound according to claim 1, in which the (C₁-C₄)alkylene group of W denotes the —(CH₂)₁₋₄-group.

12. The compound according to claim 1, of formula (I′″): 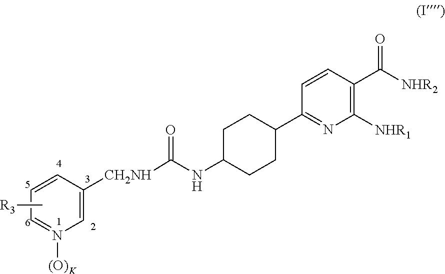
in which k is 0 or 1, R₁ represents a phenyl group optionally substituted with a trifluoromethyl group, R₂ represents a (C₁-C₆)alkyl group substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, and R₃ is positioned in position 5 or 6.

13. The compound according to claim 12, in which the cyclohexylene group is cis.

14. The compound according to claim 10 in which the (C₄-C₆)heterocycloalkyl group formed by the NRₐR_{b} group is chosen from

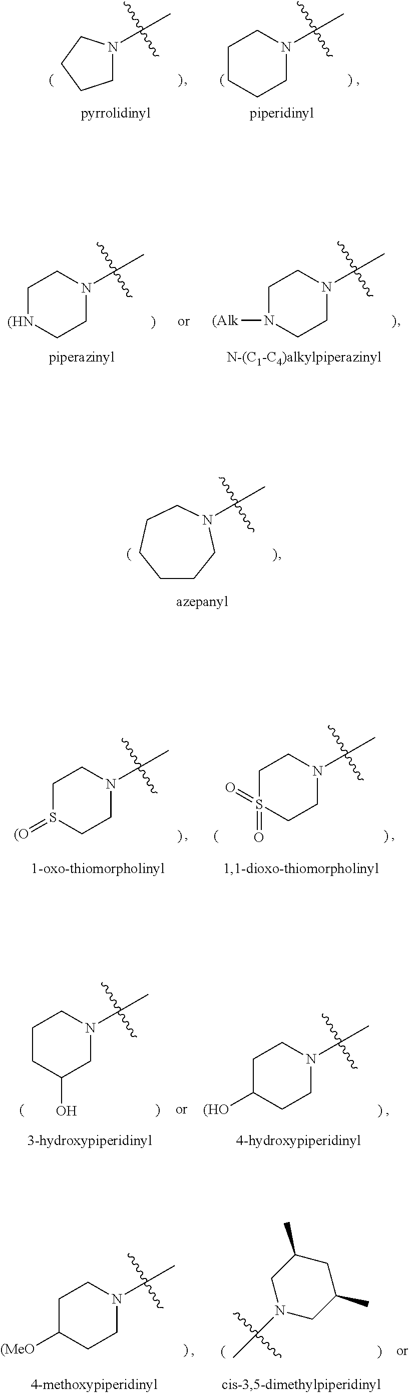

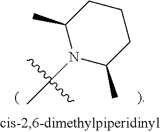

15. The compound according to claim 1 chosen from the following list:
2-ethylamino-N-methyl-6-[4-(3-pyridin-3-ylmethylureido)butyl]nicotinamide
2-cyclopropylamino-N-methyl-6-[4-(3-pyridin-3-ylmethylureido)butyl]nicotinamide
N-methyl-2-phenylamino-6-[4-(3-pyridin-3-ylmethylureido)butyl]nicotinamide
6-{4-[3-(6-aminopyridin-3-ylmethyl)ureido]butyl}-2-ethylamino-N-(2-piperidin-1-yl-ethyl)nicotinamide
6-{4-[3-(6-aminopyridin-3-ylmethyl)ureido]butyl}-2-ethylamino-N-methylnicotinamide
6-{4-[3-(6-amino-5-methylpyridin-3-ylmethyl)ureido]butyl}-2-ethylamino-N-methylnicotinamide
6-{4-[3-(6-aminopyridin-3-ylmethyl)ureido]butyl}-N-methyl-2-phenylaminonicotinamide
2-ethylamino-N-methyl-6-[4-(3-pyridin-3-ylmethylureido)but-1-ynyl]nicotinamide
2-cyclopropylamino-N-methyl-6-[4-(3-pyridin-3-ylmethylureido)but-1-ynyl]nicotinamide
N-methyl-2-phenylamino-6-[4-(3-pyridin-3-ylmethylureido)but-1-ynyl]nicotinamide
6-{4-[3-(6-aminopyridin-3-ylmethyl)ureido]butyl}-2-isopropylamino-N-methylnicotinamide
6-{4-[3-(6-dimethylaminopyridin-3-ylmethyl)ureido]butyl}-2-isopropylamino-N-methylnicotinamide
2-isopropylamino-N-methyl-6-{4-[3-(6-methylpyridin-3-ylmethyl)ureido]butyl}nicotinamide
2-isopropylamino-N-methyl-6-{4-[3-(5-methylpyridin-3-ylmethyl)ureido]butyl}nicotinamide
6-{cis-4-[3-(6-aminopyridin-3-ylmethyl)ureido]cyclohexyl}-N-(2-piperidin-1-ylethyl)-2-(3-trifluoromethylphenylamino)nicotinamide
6-[cis-4-({[(6-aminopyridin-3-yl)methyl]carbamoyl}amino)cyclohexyl]-N-[2-(1,1-dioxidothiomorpholin-4-yl)ethyl]-2-{[3-(trifluoromethyl)phenyl]amino}nicotinamide
6-{cis-4-[3-(6-aminopyridin-3-ylmethyl)ureido]cyclohexyl}-2-phenylamino-N-(2-piperidin-1-yl-ethyl)nicotinamide
6-{4-[3-(6-aminopyridin-3-ylmethyl)ureido]butyl}-2-ethylamino-N-(2-methoxyethyl)nicotinamide
6-[4-({[(6-aminopyridin-3-yl)methyl]carbamoyl}amino)butyl]-N-[2-(1,1-dioxidothiomorpholin-4-yl)ethyl]-2-(ethylamino)nicotinamide
6-{4-[3-(6-aminopyridin-3-ylmethyl)ureido]butyl}-2-phenylamino-N-(2-piperidin-1-ylethyl)nicotinamide
6-{4-[3-(6-aminopyridin-3-ylmethyl)ureido]butyl}-2-ethylamino-N-[2-(1-oxo-1 lambda 4-thiomorpholin-4-yl)ethyl]nicotinamide
6-{6-[3-(6-aminopyridin-3-ylmethyl)ureido]hexyl}-2-ethylamino-N-methylnicotinamide
6-{4-[3-(6-aminopyridin-3-ylmethyl)ureido]butyl}-2-ethylamino-N-(2-hydroxyethyl)nicotinamide
6-{4-[3-(6-aminopyridin-3-ylmethyl)ureido]butyl}-N-cyclopropyl-2-ethylaminonicotinamide
6-{4-[3-(6-aminopyridin-3-ylmethyl)ureido]butyl}-N-butyl-2-ethylaminonicotinamide
2-ethylamino-6-{4-[3-(5-fluoropyridin-3-ylmethyl)ureido]butyl}-N-methylnicotinamide
6-{3-[3-(6-aminopyridin-3-ylmethyl)ureido]-1,1-dimethylpropyl}-N-methyl-2-phenylaminonicotinamide
6-{4-[3-(6-aminopyridin-3-yl)ureido]butyl}-2-ethylamino-N-methylnicotinamide
6-(4-{3-[2-(6-aminopyridin-3-yl)ethyl]ureido}butyl)-2-ethylamino-N-methylnicotinamide
6-{(Z)-4-[3-(6-aminopyridin-3-ylmethyl)ureido]-but-1-enyl}-2-cyclopropylamino-N-methylnicotinamide
2-ethylamino-N-methyl-6-{4-[3-(1-oxypyridin-3-ylmethyl)ureido]butyl}nicotinamide
6-{4-[3-(6-cyanopyridin-3-ylmethyl)ureido]butyl}-2-ethylamino-N-methylnicotinamide
in the form of a base or of an addition salt with an acid or in the form of a hydrate or of a solvate.

16. A pharmaceutical composition comprising the compound of claim 1.

17. The pharmaceutical composition of claim 16 further comprising, at least one pharmaceutically acceptable excipient.

18. An anticancer agent comprising the compound of claim 1.

19. A method of treating or preventing cancer in a patient in need thereof comprising administering to said patient a therapeutically effective amount of the pharmaceutical composition of claim 16.
